(19) United States
(12) Patent Application Publication
(10) Pub. No.: US 2008/0051447 A1
(43) Pub. Date: Feb. 28, 2008

(54) Treatment Of Hypercholesterolemia, Hypertriglyceridemia And Cardiovascular-Related Conditions Using Phospholipase-A2 Inhibitors

(76) Inventors: Dominique Charmot, Campbell, CA (US); Jerry M. Buysse, Los Altos, CA (US); Han-Ting Chang, Livermore, CA (US); Michacl James Cope, Berkeley, CA (US); David Hui, Cincinnati, OH (US)

Correspondence Address: ILYPSA-McANDREWS;C/O MCANDREWS, HELD & MADISON STREET, 500 WEST MADISON STREET, SUITE 3400, CHICAGO, IL, 60661, US

(21) Appl. No.: 11/579,252

(22) PCT Filed: May 3, 2005
(86) PCT No.: PCT/US05/15281

§ 371 Date: May 25, 2007

(51) Int. Cl. A61K 31/404; A23L 1/00; A61P 3/00; A61K 31/015

(52) U.S. Cl. 514/419; 426/531; 514/765

(57) ABSTRACT

The present invention provides methods and compositions for the treatment of phospholipase-related conditions. In particular, the invention provides a method of treating insulin-related, weight-related conditions and/or cholesterol-related conditions in an animal subject. The method generally involves the administration of a non-absorbed and/or effluxed phospholipase A2 inhibitor that is localized in a gastrointestinal lumen.

### RELATED APPLICATION

This application claims priority to co-owned, co-pending U.S. patent application Ser. No. 10/838,879 entitled “Phospholipase Inhibitors Localized in the Gastrointestinal Lumen” filed May 3, 2004 by Hui et al.

### BACKGROUND OF THE INVENTION

Phospholipases are a group of enzymes that play important roles in a number of biochemical processes, including regulation of membrane fluidity and stability, digestion and metabolism of phospholipids, and production of intracellular messengers involved in inflammatory pathways, hemodynamic regulation and other cellular processes. Phospholipases are themselves regulated by a number of mechanisms, including selective phosphorylation, pH, and intracellular calcium levels. Phospholipase activities can be modulated to regulate their related biochemical processes, and a number of phospholipase inhibitors have been developed.

A large number of phospholipase-A2 (PL A2 or PL A₂) inhibitors are known in the art. PL A₂ inhibiting moieties include, for example, small molecule inhibitors as well as phospholipid analog and transition state analog compounds. Many such small-molecule inhibitors were developed, for example, for indications related to inflammatory states. A non-exhaustive, exemplification of known phospholipase-A2 inhibitors include the following classes: Alkynoylbenzoic, -Thiophenecarboxylic, -Furancarboxylic, and -Pyridinecarboxylic acids (e.g. see U.S. Pat. No. 5,086,067); Amide carboxylate derivatives (e.g. see WO9108737); Aminoacid esters and amide derivatives (e.g. see WO2002008189); Aminotetrazoles (e.g. see U.S. Pat. No. 5,968,963); Aryoxyacle thiazoles (e.g. see WO00034254); Azetidinones (e.g. see WO9702242); Benzenesulfonic acid derivatives (e.g. see U.S. Pat. No. 5,470,882); Benzoic acid derivatives (e.g. see JP08325154); Benzothiaphenes (e.g. see WO02000641); Benzyl alcohols (e.g. see U.S. Pat. No. 5,124,334); Benzyl phenyl pyrimidines (e.g. see WO00027824); Benzylamines (e.g. see U.S. Pat. No. 5,039,706); Cinammic acid compounds (e.g. see JP07252187); Cinnamic acid derivatives (e.g. see U.S. Pat. No. 5,578,639); Cyclohepta-indoles (e.g. see WO03016277); Ethaneamine-benzenes; Imidazolidinones, Thiazoldinones and Pyrrolidinones (e.g. see WO03031414); Indole glyoxamides (e.g. see U.S. Pat. No. 5,654,326); Indole glyoxamides (e.g. see WO9956752); Indoles (e.g. see U.S. Pat. No. 6,630,496 and WO9943672; Indoly (e.g. see WO003048122); Indoly containing sulfonamides; N-cyl-N-cinnamoylethylenediamine derivatives (e.g. see WO9603371); Naphyl acateamides (e.g. see EP77927); N-substituted glycines (e.g. see U.S. Pat. No. 5,298,652); Phosopholipid analogs (e.g. see U.S. Pat. No. 5,144,045 and U.S. Pat. No. 6,495,596); piperazines (e.g. see WO03048139); Pyridones and Pyrimidones (e.g. see WO03086400); 6-carbamoylpicolinic acid derivatives (e.g. see JP07224038); Steroids and their cyclic hydrocarbon analogs with amino-containing sidechains (e.g. see WO8702367); Trifluorobutanones (e.g. see U.S. Pat. No. 6,350,892 and US2002068722); Abietic derivatives (e.g. see U.S. Pat. No. 4,948,813); Benzyl phosphinate esters (e.g. see U.S. Pat. No. 5,504,073).

Pancreatic phospholipase A2 IB (PLA2) is thought to play a role in phospholipid digestion and processing. For example, PLA2 IB is an enzyme having activity for catabolizing phosphatidylcholine (PC) to form lysophosphatidylcholine (LPC) and free fatty acid (FFA) as reaction products. It has been reported that biliary phospholipids retard cholesterol uptake in the intestinal mucosa and that lypolysis of PC is a prerequisite for cholesterol absorption. (Rampone, A. J. and L. W. Long (1977). “The effect of phosphatidylcholine and lysophosphatidylcholine on the absorption and mucosal metabolism of oleic acid and cholesterol in vitro.” Biochim Biophys Acta 486(3): 500-10. Rampone, A. J. and C. M. Machida (1981). “Mode of action of lecithin in suppressing cholesterol absorption.” J Lipid Res 22(5): 744-52.) Further indication that phosphatidylcholine retards cholesterol absorption has been obtained in feeding studies in rats and man. For example, it has been reported that PLA2 IB catablolizing of PC within mixed micelles that carry cholesterol, bile acids, and triglycerides is an initial step for uptake of cholesterol into enterocytes. Mackay, K., J. R. Starr, et al. (1997). “Phosphatidylcholine Hydrolysis Is Required for Pancreatic Cholesterol Esterase- and Phospholipase A2-facilitated Cholesterol Uptake into Intestinal Caco-2 Cells.” Journal of Biological Chemistry 272(20): 13380-13389. It has been reported as well that PLA2 IB activity is required for full activation of pancreatic lipase/colipase-mediated triacyl glycerol hydrolysis within phospholipid-containing vesicles, another preliminary step in the absorption of triglycerides from the GI tract. (Young, S. C. and D. Y. Hui (1999). “Pancreatic lipase/colipase-mediated triacylglycerol hydrolysis is required for cholesterol transport from lipid emulsions to intestinal cells.” Biochem J 339 (Pt 3): 615-20). PLA2 IB inhibitors were shown to reduce cholesterol absorption in lymph fistula experiments in rats. (Homan, R. and B. R. Krause (1997). “Established and emerging strategies for inhibition of cholesterol absorption.” Current Pharmaceutical Design 3(1): 29-44).

More recently, a study involving mice genetically engineered to be PLA2 deficient (PLA2 (−/−) mice, also referred to herein as PLA2 knock-out mice), in which the PLA2 (−/−) mice were fed with a normal chow, indicated that the cholesterol absorption efficiency and the plasma lipid level were similar to the wild-type mice PLA2 (+/+). (Richmond, B. L., A. C. Boileau, et al. (2001). “Compensatory phospholipid digestion is required for cholesterol absorption in pancreatic phospholipase A(2)-deficient mice.” Gastroenterology 120(5): 1193-202). The same study also showed that in the PLA2 (−/−) group, intestinal PC was fully hydrolyzed even in the absence of pancreatic PLA2 activity. This study supports the observation that one or more other enzymes with phospholipase activity compensates for PLA2 activity in catalyzing phospholipids and facilitating cholesterol absorption. From this observation, one can further deduce that previously reported PLA2 inhibitors used to blunt cholesterol absorption (See, e.g., WO 96/01253 of Homan et al.) are probably non-selective (non-specific) to PLA2; that is, these inhibitors are apparently also interfering with phospholipases other than PLA2 (e.g., phospholipase B) to prevent such other enzymes for compensating for the lack of PLA2 activity. Accordingly, one can conclude that PLA2 inhibition, while necessary for reducing cholesterol absorption, is not itself sufficient to reduce cholesterol absorption in mice fed with a normal chow diet.

Further studies using PLA2 knockout mice reported a beneficial impact on diet-induced obesity and obesity-related insulin resistance in mice on a high-fat and high-cholesterol diet. (Huggins, Boileau et al. 2002). Significantly, and consistent with the earlier work of (Richmond, Boileau et al. 2001), no difference in weight gain was observed between the wild-type and PLA2 (−/−) mice maintained on a normal chow diet. However, compared to wild-type PLA2 (+/+) mice, the PLA2 (−/−) mice on high-fat/high-cholesterol diet were reported to have: reduced body weight gain over a sixteen week period, with the observed weight difference being due to increased adiposity in the wild-type mice; substantially lower fasting plasma leptin concentrations; improved glucose tolerance; and improved protection against high-fat-diet induced insulin resistance. However, it was reported that no significant differences were observed between the wild-type PLA2 (+/+) mice and the PLA2 (−/−) mice on high-fat/high-cholesterol diet with respect to plasma concentrations of free-fatty acids, cholesterol and triglycerides. Although there was evidence of increased lipid content in the stools of the PLA2 (−/−) mice, the effect did not produce overt steatorrhea, suggesting only a slight reduction in fat absorption.

Diabetes affects 18.2 million people in the United States, representing over 6% of the population. Diabetes is characterized by the inability to produce or properly use insulin. Diabetes type 2 (also called non-insulin-dependent diabetes or NIDDM) accounts for 80-90% of the diagnosed cases of diabetes and is caused by insulin resistance. Insulin resistance in diabetes type 2 prevents maintenance of blood glucose within desirable ranges, despite normal to elevated plasma levels of insulin.

Obesity is a major contributor to diabetes type 2, as well as other illnesses including coronary heart disease, osteoarthritis, respiratory problems, and certain cancers. Despite attempts to control weight gain, obesity remains a serious health concern in the United States and other industrialized countries. Indeed, over 60% of adults in the United States are considered overweight, with about 22% of these being classified as obese.

Diet also contributes to elevated plasma levels of cholesterol, including non-HDL cholesterol, as well as other lipid-related disorders. Such lipid-related disorders, generally referred to as dislipidemia, include hypercholesterolemia and hypertriglyceridemia among other indications. Non-HDL cholesterol is firmly associated with atherogenesis and its sequalea including cardiovascular diseases such as arteriosclerosis, coronary artery disease myocardial infarction, ischemic stroke, and other forms of heart disease. These together rank as the most prevalent type of illness in industrialized countries. Indeed, an estimated 12 million people in the United States suffer with coronary artery disease and about 36 million require treatment for elevated cholesterol levels.

In patients with hypercholesteremia, lowering of LDL cholesterol is among the primary targets of therapy. Hydroxymethylglutaryl-coenzym A (HMG-CoA) reductase inhibitors (“statins”) are reported to be used to reduce serum LDL cholesterol levels. However, severe and sometimes fatal adverse events, including liver failure and rhabdomyolysis (muscle condition) have been reported in connection with such use of statins. More recently, ezitimibe was introduced as a cholesterol absorption inhibitor, for use alone or in combination with statins. In patients with hypertriglyceridemia, fibrates (e.g. gemfibrozil) are used to lower high serum triglyceride concentrations. However, some patients report gastrointestinal side effects when using these drugs, and when gemfibrozil is used in combination with a statin, some patients develop significant myositis. Renal and/or liver failure or dysfunction are relative contraindications to gemfibrozil use as about 60-90% of the drug is reportedly cleared by the kidney, with the balance cleared by the liver. Notably, hypertriglyceridemia can be associatively linked with hypercholesterolemia; it has been reported that patients with triglyceride levels between 400 and 1000 mg/dl can have unwanted increases in LDL cholesterol by 10-30%. In patients with high triglycerides and low HDL cholesterol, nicotinic acid is used to increase serum HDL cholesterol and lower serum triglycerides. The main side effect is flushing of the skin in some patients. See generally, for example, Knopp, R H: Drug treatment of lipid disorders, New England Journal of Medicine 341:7 (1999) 498; Pasternak, R C et al: ACC/AHA/NHLBI Clinical Advisory on the use and safety of statins, Circulation 106 (2002) 1024; Grundy, S M et al: Implications of recent clinical trials for the National Cholesterol Education Program Adult Treatment Panel III Guidelines, Circulation 110 (2004) 227.

With the high prevalence of diabetes, obesity, and cholesterol-related conditions (including lipid disorders, generally), there remains a need for improved approaches to treat one or more of these conditions, including reducing unwanted side effects. Although a substantial number of studies have been directed to evaluating various phospholipase inhibitors for inflammatory-related indications, a relatively small effort has been directed to evaluating phospholipase-A2 inhibitors for efficacy in treating diet-related and/or metabolism-related indications, such as obesity, diabetes and cholesterol-related conditions including dislipidemia. Notably, in this regard, published in-vivo studies have reported no significant differences in levels of plasma free-fatty acids, plasma cholesterol and plasma triglycerides as compared between wild-type PLA2 (+/+) mice and the PLA2 (−/−) mice on high-fat/high-cholesterol diet. Hence, the reported literature has not heretofore established a basis for treating hypercholesterolemia and/or hypertriglyceridemia using phospholipase-A2 in patients that are members of a population at risk of obesity, insulin resistance or diabetes mellitus.

### SUMMARY OF THE INVENTION

The present invention provides methods, compositions, medicaments, foodstuffs and kits comprising phospholipase inhibitors having beneficial impact for treatment of phospholipase-related conditions, such as insulin-related conditions (e.g., diabetes), weight-related conditions (e.g., obesity) and/or cholesterol-related conditions.

One first aspect of the present invention relates to methods of treating a condition selected from the group consisting of hypercholesterolemia, hypertriglyceridemia, atherosclerosis, coronary artery disease and combinations thereof in a subject. The method comprises identifying the subject as a member of a population at risk of (i) obesity, (ii) insulin resistance, (iii) diabetes mellitus (e.g., diabetes type 2), (iv) a diet-related condition (e.g., a condition causally related to diet, including especially one or more of a high-carbohydrate-diet, a high-saccharide diet, a high-fat diet and/or a high-cholesterol diet), or (v) combinations thereof, and administering an effective amount of a phospholipase-A2 inhibitor (preferably a phospholipase-A₂ IB inhibitor) to the subject.

Another second aspect of the invention is directed to methods for modulating serum non-HDL cholesterol in a subject. The method comprises identifying the subject as a member of a population at risk of (i) obesity, (ii) insulin resistance, (iii) diabetes mellitus (e.g., diabetes type 2), (iv) a diet-related condition (e.g., a condition causally related to diet, including especially one or more of a high-carbohydrate-diet, a high-saccharide diet, a high-fat diet and/or a high-cholesterol diet), or (v) combinations thereof, and administering an effective amount of a phospholipase-A2 inhibitor (preferably a phospholipase-A₂ IB inhibitor) to the subject.

A further third aspect of the invention is directed to methods for modulating serum triglyceride in a subject. The method comprises identifying the subject as a member of a population at risk of (i) obesity, (ii) insulin resistance, (iii) diabetes mellitus (e.g., diabetes type 2), (iv) a diet-related condition (e.g., a condition causally related to diet, including especially one or more of a high-carbohydrate-diet, a high-saccharide diet, a high-fat diet and/or a high-cholesterol diet), or (v) combinations thereof, and administering an effective amount of a phospholipase-A2 inhibitor (preferably a phospholipase-A₂ IB inhibitor) to the subject.

In a fourth aspect, the invention relates to methods comprising use of a phospholipase-A₂ inhibitor (preferably a phospholipase-A₂ IB inhibitor) for manufacture of a medicament for use as a pharmaceutical for treating a condition of a subject selected from hypercholesterolemia, hypertrigliceridemia, atherosclerosis, coronary artery disease and combinations thereof, the subject being a member of a population at risk of (i) obesity, (ii) insulin resistance, (iii) diabetes mellitus (e.g., diabetes type 2), (iv) a diet-related condition (e.g., a condition causally related to diet, including especially one or more of a high-carbohydrate-diet, a high-saccharide diet, a high-fat diet and/or a high-cholesterol diet), or (v) combinations thereof.

In a fifth aspect, the invention relates to a food product composition comprising an edible foodstuff and a phospholipase-A₂ inhibitor (preferably a phospholipase-A₂ IB inhibitor. In some embodiments, the foodstuff can comprise (or can consist essentially of) a vitamin supplement and the phospholipase-A2 inhibitor.

Generally, in embodiments of the invention, including for example for embodiments relating to each of the aforementioned first through fifth aspects of the invention, the condition being treated can include at least one of hypercholesterolemia or hypertriglyceridemia, and in some embodiments, both such indications. Each of these embodiments can be used in various and specific combination, and in each permutation, with each other aspects and embodiments described above or below herein.

Generally, in embodiments of the invention, including for example for embodiments relating to each of the aforementioned first through fifth aspects of the invention, where the subject is identified as being a member of a population at risk of a diet-related condition, where the diet-related condition is a condition related to at least one of a high-carbohydrate-diet or a high-saccharide diet, together optionally with one or more of a high-fat diet and/or a high-cholesterol diet (in various permutations). Each of these embodiments can be used in various and specific combination, and in each permutation, with each other aspects and embodiments described above or below herein.

Generally, in embodiments of the invention, including for example for embodiments relating to each of the aforementioned first through fifth aspects of the invention, the phospholipase-A2 inhibitor can comprise a substituted organic compound (or including a moiety thereof) comprising a fused five-member ring and six-member ring. In preferred embodiments, the inhibitor can comprise a substituted organic compound (or including a moiety thereof) comprising a fused five-member ring and six-member ring having one or more heteroatoms (e.g., nitrogen, oxygen, sulfer) substituted within the ring structure of the five-member ring, within the ring structure of the six-member ring, or within the ring structure of each of the five-member and six-member rings, and in each case with substituent groups effective for imparting phospholipase-A2 inhibiting functionality to the compound (or moiety). In preferred embodiments, a phospholipase-A2 inhibitor or inhibiting moiety can comprise an indole-containing moiety (referred to herein interchangeably as an indole or an indole compound or an indole-moiety), such as a substituted indole moiety. Particularly-preferred indole compounds and moieties are disclosed further herein. Each of these embodiments can be used in various and specific combination, and in each permutation, with each other aspects and embodiments described above or below herein.

Generally, in embodiments of the invention, including for example for embodiments relating to each of the aforementioned first through fifth aspects of the invention, the phospholipase-A2 inhibitor can have lumen-localization functionality. For example, the phospholipase-A2 inhibitor can have chemical and physical properties that impart lumen-localization functionality to the inhibitor. Preferably in such embodiments, the inhibitors of these embodiments can have chemical and/or physical properties such that at least about 80% of the phospholipase inhibitor remains in the gastrointestinal lumen, and preferably at least about 90% of the phospholipase inhibitor remains in the gastrointestinal lumen (in each case, following administration of the inhibitor to the subject). Such chemical and/or physical properties can be realized, for example, by an inhibitor comprising at least one moiety selected from an oligomer moiety, a polymer moiety, a hydrophobic moiety, a hydrophilic moiety, a charged moiety and combinations thereof. These embodiments can be used in various and specific combination, and in each permutation, with other aspects and embodiments described above or below herein.

Generally, in embodiments of the invention, including for example for embodiments relating to each of the aforementioned first through fifth aspects of the invention, the phospholipase-A2 inhibitor can comprise or consist essentially of the substituted organic compound having a fused five-member ring and six-member ring. In some embodiments, the phospholipase inhibitor can comprise a moiety of the substituted organic compound having a fused five-member ring and six-member ring, with the moiety being linked (e.g., covalently linked, directly or indirectly using a linking moiety) to a non-absorbed or non-absorbable moiety, preferably to a non-absorbed or non-absorbable oligomer or polymer moiety. These embodiments can be used in various and specific combination, and in each permutation, with other aspects and embodiments described above or below herein.

Generally, in embodiments of the invention, including for example for embodiments relating to each of the aforementioned first through fifth aspects of the invention, the phospholipase-A₂ inhibitor does not induce substantial steatorrhea following administration or ingestion thereof. These embodiments can be used in various and specific combination, and in each permutation, with other aspects and embodiments described above or below herein.

Although various features are described above to provide a summary of various aspects of the invention, it is contemplated that many of the details thereof as described below can be used with each of the various aspects of the invention, without limitation. Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIG. 1 is a schematic representation of a chemical reaction in which phospholipase-A2 enzyme (PLA2) catalyzes hydrolysis of phospholipids to corresponding lysophospholipids.

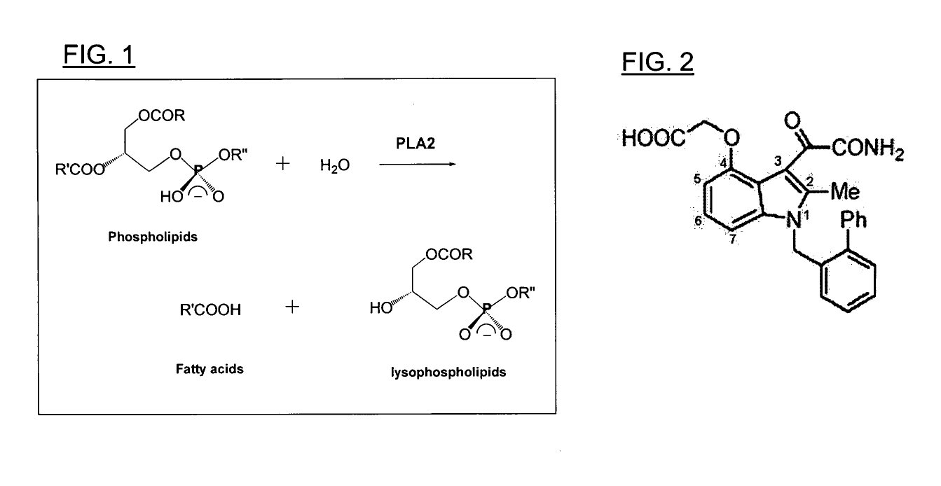

FIG. 2 is a chemical formula for [2-(3-(2-amino-2-oxoacetyl)-1-(biphenyl-2-ylmethyl)-2-methyl-1H-indol-4-yloxy)acetic acid], also referred to herein as ILY-4001 and as methyl indoxam.

FIG. 3 is a graph illustrating the results of Example 5A, showing body weight gain in groups of mice receiving ILY-4001 at low dose (4001-L) and high dose (4001-H) as compared to wild-type control group (Control) and as compared to genetically deficient PLA2 (−/−) knock-out mice (PLA2 KO).

FIG. 4 is a graph illustrating the results of Example 5B, showing fasting serum glucose levels in groups of mice receiving ILY-4001 at low dose (4001-L) and high dose (4001-H) as compared to wild-type control group (Control) and as compared to genetically deficient PLA2 (−/−) knock-out mice (PLA2 KO).

FIGS. 5A and 5B are graphs illustrating the results of Example 5C, showing serum cholesterol levels (FIG. 5A) and serum triglyceride levels (FIG. 5B) in groups of mice receiving ILY-4001 at low dose (4001-L) and high dose (4001-H) as compared to wild-type control group (Control) and as compared to genetically deficient PLA2 (−/−) knock-out mice (PLA2 KO).

FIGS. 6A through 6D are schematic representations including chemical formulas illustrating indole compounds (FIG. 6A, FIG. 6C and FIG. 6D) and indole-related compounds (FIG. 6B).

FIGS. 7A and 7B are a schematic representation (FIG. 7A) of an in-vitro fluorometric assay for evaluating PLA2 IB enzyme inhibition, and a graph (FIG. 7B) showing the results of Example 6A in which the assay was used to evaluate ILY-4001 [2-(3-(2-amino-2-oxoacetyl)-1-(biphenyl-2-ylmethyl)-2-methyl-1H-indol-4-yloxy)acetic acid].

FIGS. 8A and 8B are graphs showing the results from the in-vitro Caco-2 permeability study of Example 6B for ILY-4001 [2-(3-(2-amino-2-oxoacetyl)-1-(biphenyl-2-ylmethyl)-2-methyl-1H-indol-4-yloxy)acetic acid] (FIG. 8A) and for Lucifer Yellow and Propranolol as paracellular and transcellular transport controls (FIG. 8B).

FIG. 9 is a schematic illustration, including chemical formulas, which outlines the overall synthesis scheme for ILY-4001 [2-(3-(2-amino-2-oxoacetyl)-1-(biphenyl-2-ylmethyl)-2-methyl-1H-indol-4-yloxy)acetic acid] as described in Example 4.

### DETAILED DESCRIPTION OF THE INVENTION

The present invention provides phospholipase inhibitors, compositions (including pharmaceutical formulations, medicaments and foodstuffs) comprising such phospholipase inhibitors, methods for making such formulations, medicaments and foodstuffs, and methods for use thereof as pharmaceuticals for treatments of various conditions. The phospholipase inhibitors of the present invention can find use in treating a number of phospholipase-related conditions, including insulin-related conditions (e.g., diabetes), weight-related conditions (e.g., obesity), cholesterol-related disorders and any combination thereof, as described in detail below. In particular, phospholipase-A2 inhibitors—including especially secreted, calcium-dependent phospholipase inhibitors, and including especially phospholipase-A2 IB inhibitors—can be used advantageously for treating one or more of hypercholesterolemia, hypertriglyceridemia, atherosclerosis and coronary artery disease in certain subjects. Specifically, such phospholipase-A2 inhibitors are used for treating subjects identified as a member of a population at risk of (i) obesity, (ii) insulin resistance, (iii) diabetes mellitus (e.g., diabetes type 2), (iv) a diet-related condition or (v) combinations thereof. As used in this context, a diet related condition is preferably a condition related to diet (e.g., a condition causally related to diet), including for example one or more of a high-carbohydrate-diet, a high-saccharide diet, a high-fat diet and/or a high-cholesterol diet. In preferred embodiments, such phospholipase-A2 inhibitors are used for treating at least one of hypercholesterolemia and hypertriglyceridemia (and in some embodiments both of such indications). In preferred embodiments, the diet related condition is preferably a condition related to at least one of a high-carbohydrate-diet or a high-saccharide diet, together optionally with one or more of a high-fat diet and/or a high-cholesterol diet.

Overview

The invention comprises, in one aspect, a method of treating particular conditions—namely dislipidemia conditions including especially hypercholesterolemia and hypertriglyceridemia, in particular patients—namely, patients having a heightened risk of one or more of obesity, insulin resistance, diabetes mellitus such as diabetes type 2, and a diet-related condition.

Hepatic triglyceride synthesis is regulated by available fatty acids, glycogen stores, and an insulin versus glucagon ratio. Patients with a high glucose diet (including, for example, patients on a high-carbohydrate diet or a high-saccharide diet, alone or in combination with a high-fat diet and/or a high-cholesterol diet) and/or patients in a population known to typically consume such diets are likely to have a balance of hormones that maintains an excess of insulin and are therefore also likely to build up glycogen stores, both of which enhance hepatic triglyceride synthesis. In addition, diabetic patients are particularly susceptible, since they are often overweight and are in a state of caloric excess in view of the underlying metabolic disorder. Hence, the present invention is particularly of interest, in each embodiment herein described, with respect to treatments directed to hypertriglyceridemia.

The phospholipase A2 inhibitors of the present invention can modulate serum triglycerides and serum cholesterol. Without being bound by theory not specifically recited in the claims, such modulation may occur through more than one mechanistic path. For example, the phospholipase A2 inhibitors of the invention can modulate cholesterol absorption and triglyceride absorption from the gastrointestinal tract, and can also modulate the metabolism of fat and glucose, for example, via signaling molecules such as lysophosphatidylcholine (the reaction product of PLA2-catalyzed hydrolysis of phosphatidylcholine), where such signaling molecules operate directly and/or in conjunction with other hormones such as insulin. Such metabolic modulation can directly impact both serum cholesterol and triglyceride levels in patients that are members of the subject population (as described above), and in particular in patients on a high-disaccharide diet, on a high-carbohydrate diet, on a high-fat/high-saccharide diet, or on a high-fat/high-carbohydrate diet. In this regard, VLDL is a lipoprotein packaged by the liver for endogenous circulation from the liver to the peripheral tissues. VLDL contains triglycerides, cholesterol, and phospholipase at its core along with apolipoproteins B100, C1, CII, CIII, and E at its perimeter. Triglycerides make up more than half of VLDL by weight and the size of VLDL is determined by the amount of triglyceride. Very large VLDL is secreted by the liver in states of caloric excess, in diabetes mellitus, and after alcohol consumption, because excess triglycerides are present. Inhibition of phospholipase A2 activity can modulate metabolism, including for example hepatic triglyceride synthesis. Modulated (e.g., reduced or at least a relatively reduced increase in) triglyceride synthesis can provide a basis for modulating serum triglyceride levels and/or serum cholesterol levels, and further, can provide a basis for treating hypertriglyceridemia and/or hypercholesterolemia. Such treatments are particularly beneficial to both diabetic patients (who typically replace their carbohydrate restrictions with higher fat meals), and to hypertriglyceridemic patients (who typically substitute fat with high carbohydrate meals). In this regard, increased protein meals alone are usually not sustainable in the long term for most diabetic and/or hypertriglyceridemic patients.

Moreover, the modulation of serum triglyceride levels can have a beneficial effect on cardiovascular diseases such as atherosclerosis. Triglycerides included in VLDL packaged and released from the liver into circulation are in turn, hydrolyzed by lipoprotein lipase, such that VLDL are converted to VLDL remnants (=IDL). VLDL remnants can either enter the liver (the large ones preferentially do this) or can give rise to LDL. Hence, elevated VLDL in the circulation lowers HDL, which is responsible for reverse cholesterol transport. Since hypertriglyceridemia contributes to elevated LDL levels and also contributes to lowered HDL levels, hypertriglyceridemia is a risk factor for cardiovascular diseases such as atherosclerosis and coronary artery disease (among others, as noted above). Accordingly, modulating hypertriglyceridemia using the phospholipase-A2 inhibitors of the present invention also provide a basis for treating such cardiovascular diseases.

Methods of Treating Phospholipase-Related Conditions

The methods of the present invention, in preferred embodiments as directed toward treating hypertriglyceridemia and hypercholesterolemia, can involve modulating the activity of a phospholipase-A2 and/or modulating absorption of a phospholipase-A2 through the gastrointestinal mucosa, and/or modulating the production and/or absorption of one or more products resulting from enzymatic hydrolysis of phospholipid substrate by the phospholipase. Such methods can be used advantageously together with other methods, including for example methods broadly directed to treating insulin-related conditions (e.g., diabetes), weight-related conditions (e.g., obesity) and/or cholesterol-related conditions (including dislipidemia generally) and any combination thereof.

The present invention provides methods, pharmaceutical compositions, medicaments, and kits for the treatment of animal subjects. The term “animal subject” as used herein includes humans as well as other mammals. For example, the mammals can be selected from mice, rats, rabbits, guinea pigs, hamsters, cats, dogs, porcine, poultry, bovine and horses, as well as combinations thereof.

The term “treating” as used herein includes achieving a therapeutic benefit and/or a prophylactic benefit. By therapeutic benefit is meant eradication or amelioration of the underlying disorder being treated. For example, in a diabetic patient, therapeutic benefit includes eradication or amelioration of the underlying diabetes. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, with respect to diabetes reducing PL A₂ activity can provide therapeutic benefit not only when insulin resistance is corrected, but also when an improvement is observed in the patient with respect to other disorders that accompany diabetes like fatigue, blurred vision, or tingling sensations in the hands or feet. For prophylactic benefit, a phospholipase inhibitor of the present invention may be administered to a patient at risk of developing a phospholipase-related condition, e.g., diabetes, obesity, or hypercholesterolemia, or to a patient reporting one or more of the physiological symptoms of such conditions, even though a diagnosis may not have been made.

The present invention provides compositions comprising a phospholipase inhibitor that, in some embodiments, can be not absorbed through a gastrointestinal mucosa and/or that can be localized in a gastrointestinal lumen as a result of efflux from a gastrointestinal mucosal cell.

In preferred embodiments, the phospholipase inhibitors of the present invention produce a benefit, including either a prophylactic benefit, a therapeutic benefit, or both, in treating one or more conditions by inhibiting phospholipase-A2 activity.

In some embodiments, the conditions being treated can be induced by diet; that is, conditions can be brought on, accelerated, exacerbated, or otherwise influenced by diet. Such conditions can include, for example, but are not limited to, diabetes, weight gain, dislipidemia (e.g., hyperlipidemia, hypercholesterolemia, hypertriglyceridemia), and well-known derivative indications including cardiovascular disease (such as heart disease and stroke), hypertension, cancer sleep apnea, osteoarthritis, gallbladder disease, fatty liver disease, diabetes type 2 and other insulin-related conditions. In some embodiments, one or more of these conditions may be produced as a result of consumption of one or more of a high-carbohydrate diet, high-saccharide diet, high-fat diet or high-cholesterol diet (generally referred to alone and/or in various combinations as a Western diet). In some embodiments, however, one or more of the conditions being treated may be produced as a result of genetic causes, metabolic disorders, environmental factors, behavioral factors, or any combination of these.

Western Diets and Western-Related Diets

Generally, some embodiments of the invention relate to one or more of a high-carbohydrate diet, a high-saccharide diet, a high-fat diet and/or a high-cholesterol diet, in various combinations. Such diets are generally referred to herein as a “high-risk diets” (and can include, for example, Western diets). Such diets can heighten the risk profile of a subject patient for one or more conditions, including an obesity-related condition, an insulin-related condition and/or a cholesterol-related condition. In particular, such high-risk diets can, in some embodiments, include at least a high-carbohydrate diet together with one or more of a high-saccharide diet, a high-fat diet and/or a high-cholesterol diet. A high-risk diet can also include a high-saccharide diet in combination with one or both of a high-fat diet and/or a high-cholesterol diet. A high-risk diet can also comprise a high-fat diet in combination with a high-cholesterol diet. In some embodiments, a high-risk diet can include the combination of a high-carbohydrate diet, a high-saccharide diet and a high-fat diet. In other embodiments, a high-risk diet can include a high-carbohydrate diet, a high-saccharide diet, and a high-cholesterol diet. In other embodiments, a high-risk diet can include a high-carbohydrate diet, a high-fat diet and a high-cholesterol diet. In yet further embodiments, a high-risk diet can include a high-saccharide diet, a high-fat diet and a high-cholesterol diet. In some embodiments, a high-risk diet can include a high-carbohydrate diet, a high-saccharide diet, a high-fat diet and a high-cholesterol diet.

Generally, the diet of a subject can comprise a total caloric content, for example, a total daily caloric content. In some embodiments, the subject diet can be a high-fat diet. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat diet is combined with one or more of a high-carbohydrate diet, a high-saccharide diet or a high-cholesterol diet, at least about 15% or at least about 10% of the total caloric content can come from fat.

Similarly, in some embodiments, the diet can be a high-carbohydrate diet. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate diet is combined with one or more of a high-fat diet, a high-saccharide diet or a high-cholesterol diet, at least about 15% or at least about 10% of the total caloric content can come from carbohydrate.

Further, in some embodiments, the diet can be a high-saccharide diet. In embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide diet is combined with one or more of a high-fat diet, a high-carbohydrate diet or a high-cholesterol diet, at least about 15% or at least about 10% of the total caloric content can come from saccharides.

Similarly, in some embodiments, the diet can be a high-cholesterol diet. In such embodiments, the diet can comprise at least about 1% cholesterol (wt/wt, relative to fat). In other such embodiments, the diet can comprise at least about 0.5% or at least about 0.3% or at least about 0.1%, or at least about 0.07% cholesterol (wt/wt relative to fat). In some embodiments, in which a high-cholesterol diet is combined with one or more of a high-fat diet, a high-carbohydrate diet or a high-saccharide diet, the diet can comprise at least about 0.05% or at least about 0.03% cholesterol (wt/wt, relative to fat).

As an example, a high fat diet can include, for example, diets high in meat, dairy products, and alcohol, as well as possibly including processed food stuffs, red meats, soda, sweets, refined grains, deserts, and high-fat dairy products, for example, where at least about 25% of calories come from fat and at least about 8% come from saturated fat; or at least about 30% of calories come from fat and at least about 10% come from saturated fat; or where at least about 34% of calories came from fat and at least about 12% come from saturated fat; or where at least about 42% of calories come from fat and at least about 15% come from saturated fat; or where at least about 50% of calories come from fat and at least about 20% come from saturated fat. One such high fat diet is a “Western diet” which refers to the diet of industrialized countries, including, for example, a typical American diet, Western European diet, Australian diet, and/or Japanese diet. One particular example of a Western diet comprises at least about 17% fat and at least about 0.1% cholesterol (wt/wt); at least about 21% fat and at least about 0.15% cholesterol (wt/wt); or at least about 25% and at least about 0.2% cholesterol (wt/wt).

Such high-risk diets may include one or more high-risk foodstuffs.

Considered in the context of a foodstuff, generally, some embodiments of the invention relate to one or more of a high-carbohydrate foodstuff, a high-saccharide foodstuff, a high-fat foodstuff and/or a high-cholesterol foodstuff, in various combinations. Such foodstuffs are generally referred to herein as a “high-risk foodstuffs” (including for example Western foodstuffs). Such foodstuffs can heighten the risk profile of a subject patient for one or more conditions, including an obesity-related condition, an insulin-related condition and/or a cholesterol-related condition. In particular, such high-risk foodstuffs can, in some embodiments, include at least a high-carbohydrate foodstuff together with one or more of a high-saccharide foodstuff, a high-fat foodstuff and/or a high-cholesterol foodstuff. A high-risk foodstuff can also include a high-saccharide foodstuff in combination with one or both of a high-fat foodstuff and/or a high-cholesterol foodstuff. A high-risk foodstuff can also comprise a high-fat foodstuff in combination with a high-cholesterol foodstuff. In some embodiments, a high-risk foodstuff can include the combination of a high-carbohydrate foodstuff, a high-saccharide foodstuff and a high-fat foodstuff. In other embodiments, a high-risk foodstuff can include a high-carbohydrate foodstuff, a high-saccharide foodstuff, and a high-cholesterol foodstuff. In other embodiments, a high-risk foodstuff can include a high-carbohydrate foodstuff, a high-fat foodstuff and a high-cholesterol foodstuff. In yet further embodiments, a high-risk foodstuff can include a high-saccharide foodstuff, a high-fat foodstuff and a high-cholesterol foodstuff. In some embodiments, a high-risk foodstuff can include a high-carbohydrate foodstuff, a high-saccharide foodstuff, a high-fat foodstuff and a high-cholesterol foodstuff.

Hence, the food product composition can comprise a foodstuff having a total caloric content. In some embodiments, the food-stuff can be a high-fat foodstuff. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat foodstuff is combined with one or more of a high-carbohydrate foodstuff, a high-saccharide foodstuff or a high-cholesterol foodstuff, at least about 15% or at least about 10% of the total caloric content can come from fat.

Similarly, in some embodiments, the food-stuff can be a high-carbohydrate foodstuff. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate foodstuff is combined with one or more of a high-fat foodstuff, a high-saccharide foodstuff or a high-cholesterol foodstuff, at least about 15% or at least about 10% of the total caloric content can come from carbohydrate.

Further, in some embodiments, the food-stuff can be a high-saccharide foodstuff. In such embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide foodstuff is combined with one or more of a high-fat foodstuff, a high-carbohydrate foodstuff or a high-cholesterol foodstuff, at least about 15% or at least about 10% of the total caloric content can come from saccharides.

Similarly, in some embodiments, the food-stuff can be a high-cholesterol foodstuff. In such embodiments, the food-stuff can comprise at least about 1% cholesterol (wt/wt, relative to fat). In other such embodiments, the foodstuff can comprise at least about 0.5%, or at least about 0.3% or at least about 0.1%, or at least about 0.07% cholesterol (wt/wt relative to fat). In some embodiments, in which a high-cholesterol foodstuff is combined with one or more of a high-fat foodstuff, a high-carbohydrate foodstuff or a high-saccharide foodstuff, the foodstuff can comprise at least about 0.05% or at least about 0.03% cholesterol (wt/wt, relative to fat).

As noted above, the methods of the invention can be used advantageously together with other methods, including for example methods broadly directed to treating insulin-related conditions, weight-related conditions and/or cholesterol-related conditions (including dislipidemia generally) and any combination thereof. Aspects of such conditions are described below.

Treatment of Insulin-Related Conditions

The term “insulin-related disorders” as used herein refers to a condition such as diabetes where the body does not produce and/or does not properly use insulin. Typically, a patient is diagnosed with pre-diabetes or diabetes by using a Fasting Plasma Glucose Test (FPG) and/or an Oral Glucose Tolerance Test (OGTT). In the case of the FPG test, a fasting blood glucose level between about 100 and about 125 mg/dl can indicate pre-diabetes; while a person with a fasting blood glucose level of about 126 mg/dl or higher can indicate diabetes. In the case of the OGTT test, a patient's blood glucose level can be measured after a fast and two hours after drinking a glucose-rich beverage. A two-hour blood glucose level between about 140 and about 199 mg/dl can indicate pre-diabetes; while a two-hour blood glucose level at about 200 mg/dl or higher can indicate diabetes.

In certain embodiments, a lumen localized phospholipase inhibitor of the present invention produces a benefit in treating an insulin-related condition, for example, diabetes, preferably diabetes type 2. For example, such benefits may include, but are not limited to, increasing insulin sensitivity and improving glucose tolerance. Other benefits may include decreasing fasting blood insulin levels, increasing tissue glucose levels and/or increasing insulin-stimulated glucose metabolism.

Without being limited to any particular hypothesis, these benefits may result from a number of effects brought about by reduced PL A₂ activity, including, for example, reduced membrane transport of phospholipids across the gastrointestinal mucosa and/or reduced production of 1-acyl lysophospholipids, such as 1-acyl lysophosphatydylcholine and/or reduced transport of lysophospholipids, 1-acyl lysophosphatydylcholine, that may act as a signaling molecule in subsequent pathways involved in diabetes or other insulin-related conditions.

In some embodiments, a lumen-localized phospholipase inhibitor is used that inhibits phospholipase A2 but does not inhibit or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase.

Treatment of Weight-Related Conditions

The term “weight-related conditions” as used herein refers to unwanted weight gain, including overweight, obese and/or hyperlipidemic conditions, and in particular weight gain caused by a high fat or Western diet. Typically, body mass index (BMI) is used as the criteria in determining whether an individual is overweight and/or obese. An adult is considered overweight if, for example, he or she has a body mass index of at least about 25, and is considered obese with a BMI of at least about 30. For children, charts of Body-Mass-Index for Age are used, where a BMI greater than about the 85th percentile is considered “at risk of overweight” and a BMI greater than about the 95th percentile is considered “obese.”

In certain embodiments, a lumen localized phospholipase A2 inhibitor of the present invention can be used to treat weight-related conditions, including unwanted weight gain and/or obesity. In certain embodiments, a lumen localized phospholipase A2 inhibitor decreases fat absorption after a meal typical of a Western diet. In certain embodiments, a lumen localized phospholipase A2 inhibitor increases lipid excretion from a subject on a Western diet. In certain preferred embodiments, the phospholipase inhibitor reduces weight gain in a subject on a (typical) Western diet. In certain embodiments, practice of the present invention can preferentially reduce weight gain in certain tissues and organs, e.g., in some embodiments, a phospholipase A2 inhibitor can decrease weight gain in white fat of a subject on a Western diet.

Without being limited to any particular hypothesis, these benefits may result from a number of effects brought about by reduced PL A₂ activity. For example, inhibition of PL A₂ activity may reduce transport of phospholipids through the gastrointestinal lumen, for example, through the small intestine apical membrane, causing a depletion of the pool of phospholipids (e.g. phosphatidylcholine) in enterocytes, particularly in mammals fed with a high fat diet. In such cases, the de novo synthesis of phospholipids may not be sufficient to sustain the high turnover of phospholipids, e.g. phosphatidylcholine, needed to carry triglycerides, for example by transport in chylomicrons (See Tso, in Fat Absorption, 1986, chapt. 6 177-195, Kuksis A., Ed.), incorporated herein by reference.

PL A₂ inhibition can also reduce production of 1-acyl lysophospholipids, such as 1-acyl lysophosphatydylcholine, that may act as a signaling molecule in subsequent up-regulation pathways of fat absorption, including, for example the release of additional digestive enzymes or hormones, e.g., secretin. See, Huggins, Protection against diet-induced obesity and obesity-related insulin resistance in Group 1B—PL A₂-deficient mice, Am. J. Physiol. Endocrinol. Metab. 283:E994-E1001 (2002), incorporated herein by reference.

Another aspect of the present invention provides composition, kits and methods for reducing or delaying the onset of diet-induced diabetes through weight gain. An unchecked high fat diet can produce not only weight gain, but also can contribute to diabetic insulin resistance. This resistance may be recognized by decreased insulin and leptin levels in a subject. The phospholipase inhibitors, compositions, kits and methods disclosed herein can be used in the prophylactic treatment of diet-induced diabetes, or other insulin-related conditions, e.g. in decreasing insulin and/or leptin levels in a subject on a Western diet.

In some embodiments, a lumen-localized phospholipase inhibitor is used that inhibits phospholipase A2 but does not inhibitor or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase B.

Treatment of Cholesterol-Related Conditions

The term “cholesterol-related conditions” as used herein refers generally to a condition in which modulating the activity of HMG-CoA reductase is desirable and/or modulating the production and/or effects of one or more products of HMG-CoA reductase is desirable, and can in any case, include dislipidemia generally. In preferred embodiments, a phospholipase inhibitor of the present invention reduces the activity of HMG-CoA reductase and/or reduces the production and/or effects of one or more products of HMG-CoA reductase. For example, a cholesterol-related condition may involve elevated levels of cholesterol, in particular, non-HDL cholesterol in plasma (e.g., elevated levels of LDL cholesterol and/or VLDL/LDL levels). Typically, a patient is considered to have high or elevated cholesterol levels based on a number of criteria, for example, see Pearlman B L, The New Cholesterol Guidelines, Postgrad Med, 2002; 112(2):13-26, incorporated herein by reference. Guidelines include serum lipid profiles, such as LDL compared with HDL levels.

Examples of cholesterol-related conditions include hypercholesterolemia, lipid disorders such as hyperlipidemia, and atherogenesis and its sequelae of cardiovascular diseases, including atherosclerosis, other vascular inflammatory conditions, myocardial infarction, ischemic stroke, occlusive stroke, and peripheral vascular diseases, as well as other conditions in which decreasing cholesterol can produce a benefit.

Other cholesterol-related conditions treatable with compositions, kits, and methods of the present invention include those currently treated with statins, as well as other conditions in which decreasing cholesterol absorption can produce a benefit.

In certain embodiments, a lumen-localized phospholipase inhibitor of the present invention can be used to reduce cholesterol levels, in particular non-HDL plasma cholesterol levels, as well as to treat hypertriglyceridemia.

In some preferred embodiments, the composition can inhibit phospholipase A2 and at least one other gastrointestinal phospholipase in addition to phospholipase A2, such as preferably phospholipase B, and also such as phospholipase A1, phospholipase C, and/or phospholipase D.

In other embodiments of the invention, the differential activities of phospholipases can be used to treat certain phospholipase-related conditions without undesired side effects resulting from inhibiting other phospholipases. For example, in certain embodiments, a phospholipase inhibitor that inhibits PL A₂, but not inhibiting or not significantly inhibiting or essentially not inhibiting, for example, PLA1, PLB, PLC, or PLD can be used to treat an insulin-related condition (e.g. diabetes) and/or a weight-related condition (e.g. obesity) without affecting, or without significantly affecting, or without essentially effecting, cholesterol absorption of a subject receiving phospholipase inhibiting treatment, e.g., when the subject is on a high fat diet.

The phospholipase inhibitors, methods, and kits disclosed herein can be used in the treatment of phospholipase-related conditions. In some preferred embodiments, these effects can be realized without a change in diet and/or activity on the part of the subject. For example, the activity of PL A₂ in the gastrointestinal lumen may be inhibited to result in a decrease in fat absorption and/or a reduction in weight gain in a subject on a Western diet compared to if the subject was not receiving PL A₂ inhibiting treatment. More preferably, this decrease and/or reduction occurs without a change, without a significant change, or essentially without a change, in energy expenditure and/or food intake on the part of the subject, and without a change, or without a significant change, or essentially without a change in the body temperature of the subject. Further, in preferred embodiments, a phospholipase inhibitor of the present invention can be used to offset certain negative consequences of high fat diets without affecting normal aspects of metabolism on non-high fat diets.

The present invention also includes kits that can be used to treat phospholipase-related conditions, preferably phospholipase A2-related conditions or phospholipase-related conditions induced by diet, including, but not limited to, insulin-related conditions (e.g., diabetes, particularly diabetes type 2), weight-related conditions (e.g., obesity) and/or cholesterol-related conditions. These kits comprise at least one composition of the present invention and instructions teaching the use of the kit according to the various methods described herein.

Inhibitor Formulations, Routes of Administration, and Effective Doses

The phospholipase inhibitors useful in the present invention, or pharmaceutically acceptable salts thereof, can be delivered to a patient using a number of routes or modes of administration. The term “pharmaceutically acceptable salt” means those salts which retain the biological effectiveness and properties of the compounds used in the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxyl group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

If necessary or desirable, the phospholipase inhibitor may be administered in combination with one or more other therapeutic agents. The choice of therapeutic agent that can be co-administered with a composition of the invention will depend, in part, on the condition being treated. For example, for treating obesity, or other weight-related conditions, a phospholipase inhibitor of some embodiments of the present invention can be used in combination with a statin, a fibrate, a bile acid binder, an ezitimibe (e.g., Zetia, etc), a saponin, a lipase inhibitor (e.g. Orlistat, etc), and/or an appetite suppressant, and the like. With respect to treating insulin-related conditions, e.g., diabetes, a phospholipase inhibitor of some embodiments the present invention can be used in combination with a biguanide (e.g., Metformin), thiazolidinedione, and/or α-glucosidase inhibitor, and the like.

The phospholipase inhibitors (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries which facilitate processing of the active compounds into preparations which can be used pharmaceutically. Proper formulation is dependent upon the route of administration chosen.

The phospholipase inhibitors can be administered by direct placement, orally, and/or rectally. Preferably, the phospholipase inhibitor or the pharmaceutical composition comprising the phospholipase inhibitor is administered orally. The oral form in which the phospholipase inhibitor is administered can include a powder, tablet, capsule, solution, or emulsion. The effective amount can be administered in a single dose or in a series of doses separated by appropriate time intervals, such as hours.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions, wafers, and the like, for oral ingestion by a patient to be treated. In some embodiments, the inhibitor may be formulated as a sustained release preparation. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. In some embodiments, the oral formulation does not have an enteric coating.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

Suitable carriers used in formulating liquid dosage forms for oral as well as parenteral administration include non-aqueous, pharmaceutically-acceptable polar solvents such as hydrocarbons, alcohols, amides, oils, esters, ethers, ketones, and/or mixtures thereof, as well as water, saline solutions, electrolyte solutions, dextrose solutions (e.g., DW5), and/or any other aqueous, pharmaceutically acceptable liquid.

Suitable nonaqueous, pharmaceutically-acceptable polar solvents include, but are not limited to, alcohols (e.g., aliphatic or aromatic alcohols having 2-30 carbon atoms such as methanol, ethanol, propanol, isopropanol, butanol, t-butanol, hexanol, octanol, benzyl alcohol, amylene hydrate, glycerin (glycerol), glycol, hexylene glycol, lauryl alcohol, cetyl alcohol, stearyl alcohol, tetrahydrofurfuryl alcohol, fatty acid esters of fatty alcohols such as polyalkylene glycols (e.g., polyethylene glycol and/or polypropylene glycol), sorbitan, cholesterol, sucrose and the like); amides (e.g., dimethylacetamide (DMA), benzyl benzoate DMA, N,N-dimethylacetamide amides, 2-pyrrolidinone, polyvinylpyrrolidone, 1-methyl-2-pyrrolidinone, and the like); esters (e.g., 2-pyrrolidinone, 1-methyl-2-pyrrolidinone, acetate esters (such as monoacetin, diacetin, and triacetin and the like), and the like, aliphatic or aromatic esters (such as dimethylsulfoxide (DMSO), alkyl oleate, ethyl caprylate, ethyl benzoate, ethyl acetate, octanoate, benzyl benzoate, benzyl acetate, esters of glycerin such as mono, di, or tri-glyceryl citrates or tartrates, ethyl carbonate, ethyl oleate, ethyl lactate, N-methyl pyrrolidinone, fatty acid esters such as isopropyl myristrate, fatty acid esters of sorbitan, glyceryl monostearate, glyceride esters such as mono, di, or tri-glycerides, fatty acid derived PEG esters such as PEG-hydroxystearate, PEG-hydroxyoleate, and the like, pluronic 60, polyoxyethylene sorbitol oleic polyesters, polyoxyethylene sorbitan esters such as polyoxyethylene-sorbitan monooleate, polyoxyethylene-sorbitan monostearate, polyoxyethylene-sorbitan monolaurate, polyoxyethylene-sorbitan monopalmitate, alkyleneoxy modified fatty acid esters such as polyoxyl 40 hydrogenated castor oil and polyoxyethylated castor oils, saccharide fatty acid esters (i.e., the condensation product of a monosaccharide, disaccharide, or oligosaccharide or mixture thereof with a fatty acid(s) (e.g., saturated fatty acids such as caprylic acid, myristic acid, palmitic acid, capric acid, lauric acid, and stearic acid, and unsaturated fatty acids such as palmitoleic acid, oleic acid, elaidic acid, erucic acid and linoleic acid)), or steroidal esters and the like); alkyl, aryl, or cyclic ethers (e.g., diethyl ether, tetrahydrofuran, diethylene glycol monoethyl ether, dimethyl isosorbide and the like); glycofurol (tetrahydrofurfuryl alcohol polyethylene glycol ether); ketones (e.g., acetone, methyl isobutyl ketone, methyl ethyl ketone and the like); aliphatic, cycloaliphatic or aromatic hydrocarbons (e.g., benzene, cyclohexane, dichloromethane, dioxolanes, hexane, n-hexane, n-decane, n-dodecane, sulfolane, tetramethylenesulfoxide, tetramethylenesulfon, toluene, tetramethylenesulfoxide dimethylsulfoxide (DMSO) and the like); oils of mineral, animal, vegetable, essential or synthetic origin (e.g., mineral oils such as refined paraffin oil, aliphatic or wax-based hydrocarbons, aromatic hydrocarbons, mixed aliphatic and aromatic based hydrocarbons, and the like, vegetable oils such as linseed, soybean, castor, rapeseed, coconut, tung, safflower, cottonseed, groundnut, palm, olive, corn, corn germ, sesame, persic, peanut oil, and the like, as well as glycerides such as mono-, di- or triglycerides, animal oils such as cod-liver, haliver, fish, marine, sperm, squalene, squalane, polyoxyethylated castor oil, shark liver oil, oleic oils, and the like); alkyl or aryl halides e.g., methylene chloride; monoethanolamine; trolamine; petroleum benzin; omega-3 polyunsaturated fatty acids (e.g., α-linolenic acid, docosapentaenoic acid, docosahexaenoic acid, eicosapentaenoic acid, and the like); polyglycol ester of 12-hydroxystearic acid; polyethylene glycol; polyoxyethylene glycerol, and the like.

Other pharmaceutically acceptable solvents that can be used in formulating pharmaceutical compositions of a phospholipase inhibitor of the present invention including, for example, for direct placement, are well known to those of ordinary skill in the art, e.g. see Modern Pharmaceutics, (G. Banker et al., eds., 3d ed.) (Marcel Dekker, Inc., New York, N.Y., 1995), The Handbook of Pharmaceutical Excipients, (American Pharmaceutical Association, Washington, D.C.; The Pharmacological Basis of Therapeutics, (Goodman & Gilman, McGraw Hill Publishing), Remington's Pharmaceutical Sciences (A. Gennaro, ed., 19th ed.) (Mack Publishing, Easton, Pa., 1995), Pharmaceutical Dosage Forms, (H. Lieberman et al., eds.,) (Marcel Dekker, Inc., New York, N.Y., 1980); and The United States Pharmacopeia 24, The National Formulary 19, (National Publishing, Philadelphia, Pa., 2000).

Formulations for rectal administration may be prepared in the form of a suppository, an ointment, an enema, a tablet, or a cream for release of the phospholipase inhibitor in the gastrointestinal tract, e.g., the small intestine. Rectal suppositories can be made by mixing one or more phospholipase inhibitors of the present invention, or pharmaceutically acceptable salts thereof, with acceptable vehicles, for example, cocoa butter, with or without the addition of waxes to alter melting point. Acceptable vehicles can also include glycerin, salicylate and/or polyethylene glycol, which is solid at normal storage temperature, and a liquid at those temperatures suitable to release the phospholipase inhibitor inside the body, such as in the rectum. Oils may also be used in rectal formulations of the soft gelatin type and in suppositories. Water soluble suppository bases, such as polyethylene glycols of various molecular weights, may also be used. Suspension formulations may be prepared that use water, saline, aqueous dextrose and related sugar solutions, and glycerols, as well as suspending agents such as pectins, carbomers, methyl cellulose, hydroxypropyl cellulose or carboxymethyl cellulose, as well as buffers and preservatives.

Pharmaceutical compositions suitable for use in the present invention include compositions wherein the active ingredients are present in an effective amount, i.e., in an amount sufficient to produce a therapeutic and/or a prophylactic benefit in at least one condition being treated. The actual amount effective for a particular application will depend on the condition being treated and the route of administration. Determination of an effective amount is well within the capabilities of those skilled in the art, especially in light of the disclosure herein. For example, the IC50 values and ranges provided in Table 1 above provide guidance to enable one of ordinary skill in the art to select effective dosages of the corresponding phospholipase inhibiting moieties.

The effective amount when referring to a phospholipase inhibitor will generally mean the dose ranges, modes of administration, formulations, etc., that have been recommended or approved by any of the various regulatory or advisory organizations in the medical or pharmaceutical arts (eg, FDA, AMA) or by the manufacturer or supplier. Effective amounts of phospholipase inhibitors can be found, for example, in the Physicians Desk Reference. The effective amount when referring to producing a benefit in treating a phospholipase-related condition, such as insulin-related conditions (e.g., diabetes), weight-related conditions (e.g., obesity), and/or cholesterol related-conditions will generally mean the levels that achieve clinical results recommended or approved by any of the various regulatory or advisory organizations in the medical or pharmaceutical arts (eg, FDA, AMA) or by the manufacturer or supplier.

A person of ordinary skill using techniques known in the art can determine the effective amount of the phospholipase inhibitor. In the present invention, the effective amount of a phospholipase inhibitor localized in the gastsrointestinal lumen can be less than the amount administered in the absence of such localization. Even a small decrease in the amount of phospholipase inhibitor administered is considered useful for the present invention. A significant decrease or a statistically significant decrease in the effective amount of the phospholipase inhibitor is particularly preferred. In some embodiments of the invention, the phospholipase inhibitor reduces activity of phospholipase to a greater extent compared to non-lumen localized inhibitors. Lumen-localization of the phospholipase inhibitor can decrease the effective amount necessary for the treatment of phospholipase-related conditions, such as insulin-related conditions (e.g., diabetes), weight-related conditions (e.g., obesity) and/or cholesterol-related conditions by about 5% to about 95%. The amount of phospholipase inhibitor used could be the same as the recommended dosage or higher than this dose or lower than the recommended dose.

In some embodiments, the recommended dosage of a phospholipase inhibitor is between about 0.1 mg/kg/day and about 1,000 mg/kg/day. The effective amount for use in humans can be determined from animal models. For example, a dose for humans can be formulated to achieve circulating and/or gastrointestinal concentrations that have been found to be effective in animals, e.g. a mouse model as the ones described in the samples below.

A person of ordinary skill in the art can determine phospholipase inhibition by measuring the amount of a product of a phospholipase, e.g., lysophosphatidylcholine (LPC), a product of PL A₂. The amount of LPC can be determined, for example, by measuring small intestine, lymphatic, and/or serum levels post-prandially. Another technique for determining amount of phospholipase inhibition involves taking direct fluid samples from the gastrointestinal tract. A person of ordinary skill in the art would also be able to monitor in a patient the effect of a phospholipase inhibitor of the present invention, e.g., by monitoring cholesterol and/or triglyceride serum levels. Other techniques would be apparent to one of ordinary skill in the art. Other approaches for measuring phospholipase inhibition and/or for demonstrating the effects of phospholipase inhibitors of some embodiments are further illustrated in the examples below.

Preferred Indole-Related Compounds and Indole Compounds as PLA2 Inhibitors

In preferred embodiments, the phospholipase-A₂ IB inhibitor comprises a substituted organic compound having a fused five-member ring and six-member ring. The invention also contemplates, in another aspect, a method for modulating the metabolism of fat, glucose or cholesterol in a subject by administering an effective amount of such phospholipase-A₂ IB inhibitor to the subject. The invention includes as well, in a further aspect, methods of using a phospholipase-A₂ IB inhibitor for manufacture of a medicament, where the medicament is indicated for use as a pharmaceutical for treating a condition of a subject (e.g., a weight-related condition, an insulin-related condition, a cholesterol-related condition and combinations thereof), and where the phospholipase-A₂ IB inhibitor comprises a substituted organic compound having a fused five-member ring and six-member ring. The invention can include, moreover in another aspect, a food product composition comprising an edible foodstuff and a phospholipase-A₂ IB inhibitor, preferably where the phospholipase-A₂ IB inhibitor comprises the substituted organic compound having a fused five-member ring and six-member ring.

Hence, in generally preferred embodiments of the various aspects of the invention, the phospholipase inhibitor (or inhibiting moiety) can comprise a substituted organic compound (or moiety derived from a substituted organic compound) having a fused five-member ring and six-member ring (or as a pharmaceutically-acceptable salt thereof). Preferably, the inhibitor also comprises substituent groups effective for imparting phospholipase-A2 inhibiting functionality to the inhibitor (or inhibiting moiety), and preferably phospholipase-A2 IB inhibiting functionality. Preferably the phospholipase inhibitor a fused five-member ring and six-member ring having one or more heteroatoms (e.g., nitrogen, oxygen, sulfer) substituted within the ring structure of the five-member ring, within the ring structure of the six-member ring, or within the ring structure of each of the five-member and six-member rings (or as a pharmaceutically-acceptable salt thereof). Again preferably, the inhibitor (or inhibiting moiety) can comprise substituent groups effective for imparting phospholipase inhibiting functionality to the moiety.

As demonstrated in Example 5 (including related Examples 5A through 5C), substituted organic compounds (or moieties derived therefrom) having such fused five-member ring and six-member ring are effective phospholipase-2A IB inhibitors, with phenotypic effects approaching and/or comparable to the effect of genetically deficient PLA2 (−/−) mice. Moreover, such compound (or moieties derived therefrom) are effective in treating conditions such as weight-related conditions, insulin-related conditions, and cholesterol-related conditions, including in particular conditions such as obesity, diabetes mellitus, insulin resistance, glucose intolerance, hypercholesterolemia and hypertriglyceridemia.

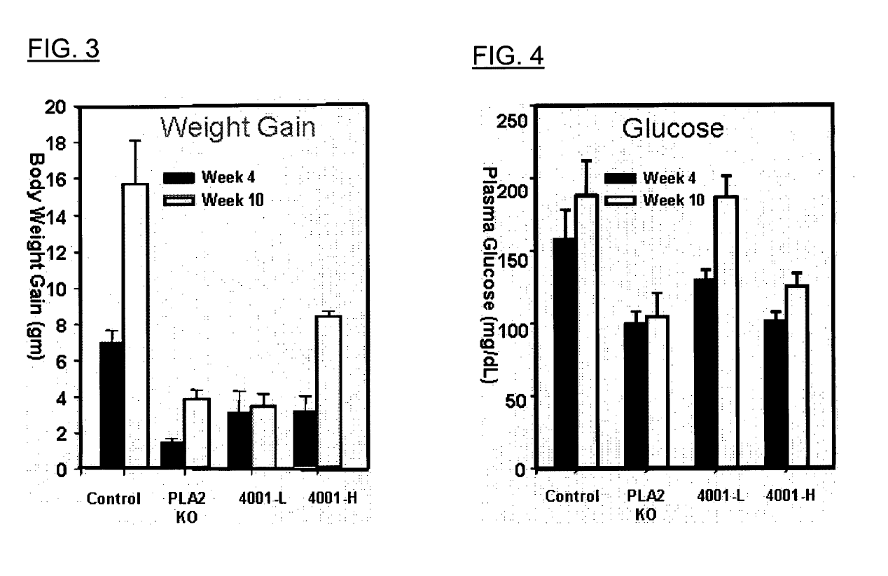

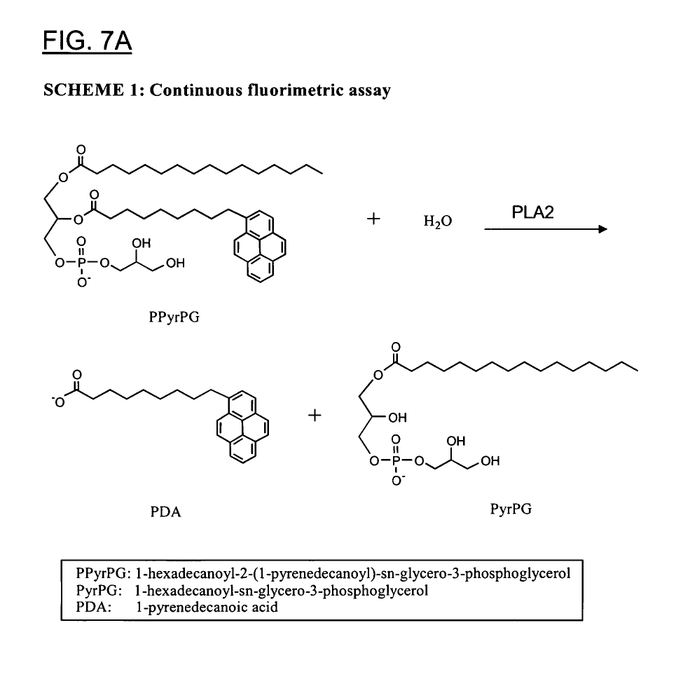

Although a particular compound was evaluated in-vivo in the study described in Example 5, namely the compound 2-(3-(2-amino-2-oxoacetyl)-1-(biphenyl-2-ylmethyl)-2-methyl-1H-indol-4-yloxy)acetic acid, shown in FIG. 2, the results of this study support a more broadly-defined invention, because the inhibitive effect can be realized and understood through structure-activity-relationships as described in detail hereinafter. Briefly, without being bound by theory not specifically recited in the claims, compounds comprising the fused five-membered and six-membered rings have a structure that advantageously provides an appropriate bond-length and bond-angles for positioning substituent groups—for example at positions 3 and 4 of an indole-compound as represented in FIG. 6A, and at the-R₃ and-R₄ positions of the indole-related compounds comprising fused five-membered and six-membered rings as represented in FIG. 6B. Mirror-image analogues of such indole compounds and of such indole-related compounds also can be used in connection with this invention, as described below.

In some preferred embodiments, described below, the phospholipase-A2 inhibitor (or inhibiting moiety) can comprise indole compounds or indole-related compounds.

Also, in some preferred embodiments, described below, the phospholipase-A2 inhibitor (or inhibiting moiety) can be a lumen-localized phospholipase-A2 inhibitor.

In particularly preferred embodiments, the phospholipase-A2 inhibiting moiety can comprise a fused five-membered ring and six-membered ring as a compound (or as a pharmaceutically-acceptable salt thereof), represented by the following formula (I): 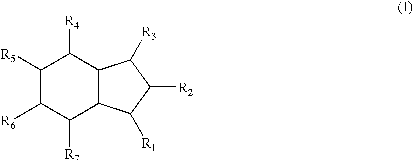
wherein the core structure can be saturated (as shown above) or unsaturated (not shown), and wherein R₁ through R₇ are independently selected from the group consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof; and additionally or alternatively, wherein R₁ through R₇ can optionally comprise, independently selected additional rings between two adjacent substitutents, with such additional rings being independently selected 5-, 6-, and/or 7-member rings which are carbocyclic rings, heterocyclic rings, and combinations thereof.

As used generally herein, including as used in connection with R₁ through R₇ in the indole-related compound shown above:

an amine group can include primary, secondary and tertiary amines;

a halide group can include fluoro, chloro, bromo, or iodo;

a carbonyl group can be a carbonyl moiety having a further substitution (defined below) as represented by the formula

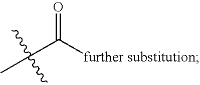

an acidic group can be an organic group as a proton donor and capable of hydrogen bonding, non-limiting examples of which include carboxylic acid, sulfate, sulfonate, phosphonates, substituted phosphonates, phosphates, substituted phosphates, 5-tetrazolyl,

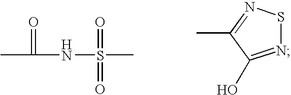

an alkyl group by itself or as part of another substituent can be a substituted or unsubstituted straight or branched chain hydrocarbon such as methyl, ethyl, n-propyl, isopropyl, n-butyl, tertiary butyl, sec-butyl, n-pentyl, n-hexyl, decyl, dodecyl, or octadecyl;

an alkenyl group by itself or in combination with other group can be a substituted or unsubstituted straight chain or branched hydrocarbon containing unsaturated bonds such as vinyl, propenyl, crotonyl, isopentenyl, and various butenyl isomers;

a carbocyclic group can be a substituted or unsubstituted, saturated or unsaturated, 5- to 14-membered organic nucleus whose ring forming atoms are solely carbon atoms, including cycloalkyl, cycloalkenyl, phenyl, spiro[5.5]undecanyl, naphthyl, norbornanyl, bicycloheptadienyl, toluoyl, xylenyl, indenyl, stilbenzyl, terphenylyl, diphenylethylenyl, phenyl-cyclohexenyl, acenaphthylenyl, and anthracenyl, biphenyl, and bibenzylyl;

a heterocyclic group can be monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur, including pyrrolyl, pyrrolodinyl, piperidinyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, dibenzothiophenyl, indazolyl, imidazo pyridinyl, benzotriazolyl, anthranilyl, 1,2-benzisoxazolyl, benzoxazolyl, benzothiazolyl, purinyl, pyridinyl, dipyridylyl. phenylpyridinyl, benzylpyridinyl, pyrimidinyl, phenylpyrimidinyl, pyrazinyl, 1,3,5-triazinyl, quinolinyl, phthalazinyl, quinazolinyl, morpholino, thiomorpholino, homopiperazinyl, tetrahydrofuranyl, tetrahydropyranyl, oxacanyl, 1,3-dioxolanyl, 1,3-dioxanyl, 1,4-dioxanyl, tetrahydrothiopheneyl, pentamethylenesulfadyl, 1,3-dithianyl, 1,4-dithianyl, 1,4-thioxanyl, azetidinyl, hexamethyleneiminium, heptamethyleneiminium, piperazinyl and quinoxalinyl;

an acylamino group can be an acylamino moiety having two further substitutions (defined below) as represented by the formula:

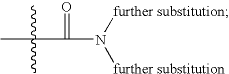

an oximyl group can be an oximyl moiety having two further substitutions (defined below) as represented by the formula:

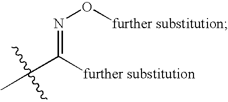

a hydrazyl group can be a hydrazyl moiety having three further substitutions (defined below) as represented by the formula:

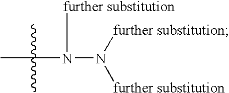

a substituted substitution group combines one or more of the listed substituent groups, preferably through moieties that include for example

an -oxygene-alkyl-acidic moiety such as

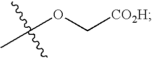

a -carbonyl-acylamino-hydrogen moiety such as

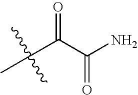

an -alkyl-carbocyclic-alkenyl moiety such as

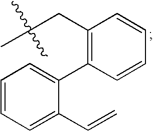

a -carbonyl-alkyl-thiol moiety such as

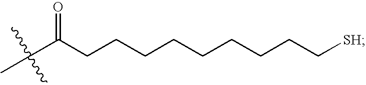

an -amine-carbonyl-amine moiety such as 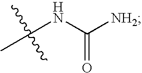
and

a further substitution group can mean a group selected from hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

Particularly preferred substituent groups R₁ through R₇ for such indole-related compounds are described below in connection with preferred indole-compounds.

In preferred embodiments, the phospholipase-A2 inhibiting moiety can comprise an indole compound (e.g., an indole-containing compound or compound containing an indole moiety), such as a substituted indole moiety. For example, in such embodiment, the indole-containing compound can be a compound represented by the formulas II, III (considered left to right as shown): 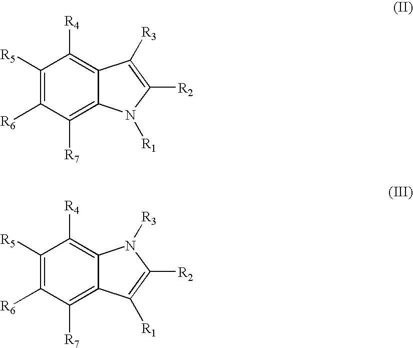
wherein R₁ through R₇ are independently selected from the groups consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof; and additionally or alternatively, wherein R₁ through R₇ can optionally, and independently form additional rings between two adjacent substitutents with such additional rings being 5-, 6-, and 7-member ring selected from the group consistin of carbocyclic rings, heterocyclic rings and combinations thereof.

Some indole compounds having additional rings include, for example, those compounds represented as formulas IVa through IVf (considered left to right in top row as IVa, IVb, IVc, and considered left to right bottom row as IVd, IVe and IVf, as shown):

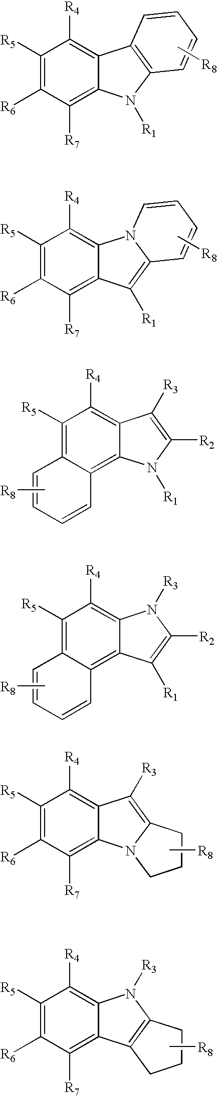

Generally, the various types of substituent groups, including carbonyl, acidic, alkyl, alkenyl, carbocyclic, heterocyclic, acylamino, oximyl, hydrazyl, substituted substitution group, can be as defined above in connection with the indole-related compounds having fused five-membered and six-membered rings.

In each of the embodiments of the invention, including for those compounds that are indole-related compounds having fused five-membered and six-membered rings, and for the indole compounds, preferred substitutent groups can be as described in the following paragraphs.

Preferred R₁ is selected from the following groups: hydrogen, oxygen, sulfur, amine, halide, hydroxyl (—OH), thiol (—SH), carbonyl, acidic, alkyl, alkenyl, carbocyclic, heterocyclic, substituted substitution group and combinations thereof. Particularly preferred R₁ is selected from the following groups: hydrogen, halide, thiol (—SH), carbonyl, acidic, alkyl, alkenyl, carbocyclic, substituted substitution group and combinations thereof. R₁ is especially preferably selected from the group consisting of alkyl, carbocyclic and substituted substitution group. The substituted substitution group for R₁ are especially preferred compounds or moieties such as:

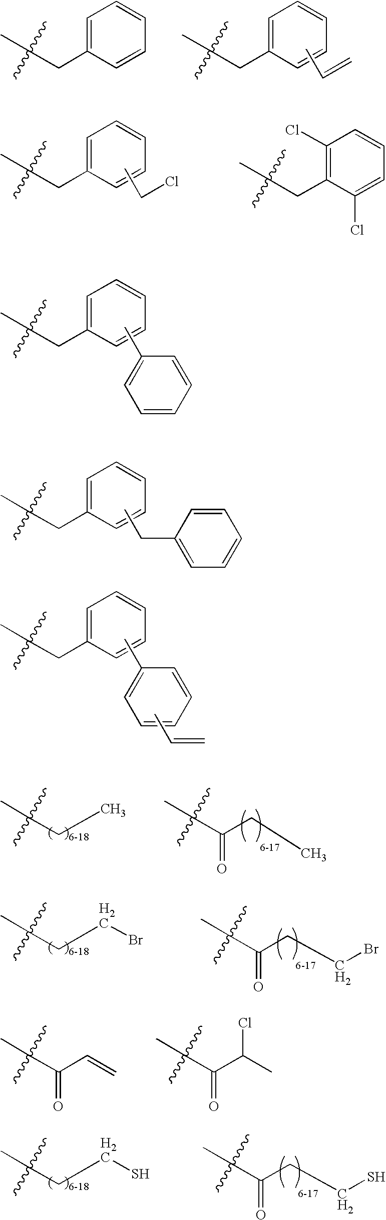

Preferred R₂ is selected from the following groups: hydrogen, oxygen, halide, carbonyl, alkyl, alkenyl, carbocyclic, substituted substitution group, and combinations thereof. Particularly preferred R₂ is selected from the following groups: hydrogen, halide, alkyl, alkenyl, carbocyclic, substituted substitution group, and combinations thereof. R₂ is preferably selected from the group consisting of halide, alkyl and substituted substitution group. The substituted substitution group for R₂ are especially preferred compounds or moieties such as:

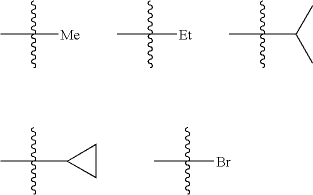

Preferred R₃ is selected from the following groups: hydrogen, oxygen, sulfur, amine, hydroxyl (—OH), thiol (—SH), carbonyl, acidic, alkyl, heterocyclic, acylamino, oximyl, hydrazyl, substituted substitution group and combinations thereof. Particularly preferred R₃ is selected from the following groups: hydrogen, oxygen, amine, hydroxyl (—OH), carbonyl, alkyl, acylamino, oximyl, hydrazyl, substituted substitution group and combinations thereof. R₃ is preferably selected from the group consisting of carbonyl, acylamino, oximyl, hydrazyl, and substituted substitution group. The substituted substitution group for R₃ are especially preferred compounds or moieties such as:

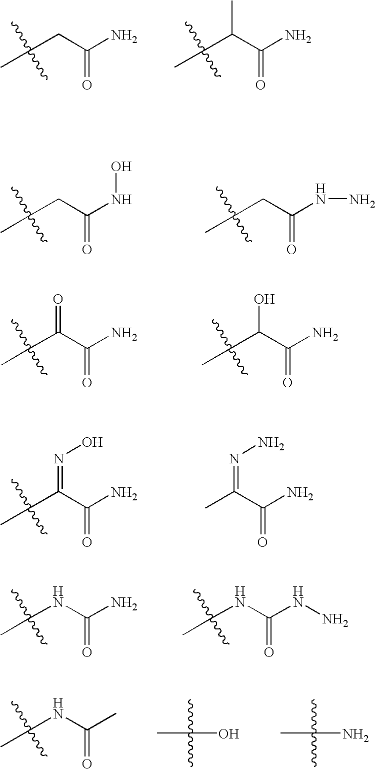

Preferred R₄ and R₅ are independently selected from the following groups: hydrogen, oxygen, sulfur, phosphorus, amine, hydroxyl (—OH), thiol (—SH), carbonyl, acidic, alkyl, alkenyl, heterocyclic, acylamino, oximyl, hydrazyl, substituted substitution group and combinations thereof. Particularly preferred R₄ and R₅ are independently selected from the following groups: hydrogen, oxygen, sulfur, amine, acidic, alkyl, substituted substitution group and combinations thereof. R₄ and R₅ are each preferably independently selected from the group consisting of oxygen, hydroxyl (—OH), acidic, alkyl, and substituted substitution group. The substituted substitution group for R₄ and for R₅ are especially preferred compounds or moieties such as:

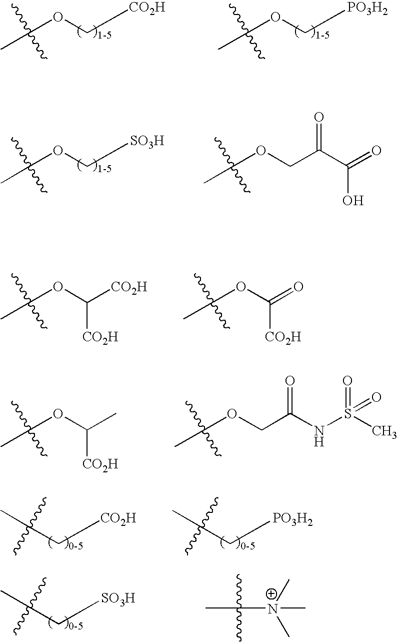

Preferred R₆ is selected from the following groups hydrogen, oxygen, amine, halide, hydroxyl (—OH), acidic, alkyl, carbocyclic, acylamino, substituted substitution group and combinations thereof. Particularly preferred R₆ is selected from the following groups: hydrogen, oxygen, amine, halide, hydroxyl (—OH), acidic, alkyl, acylamino, substituted substitution group and combinations thereof. R₆ is preferably selected from the group consisting of amine, acidic, alkyl, and substituted substitution group. The substituted substitution group for R₆ are especially preferred compounds or moieties such as:

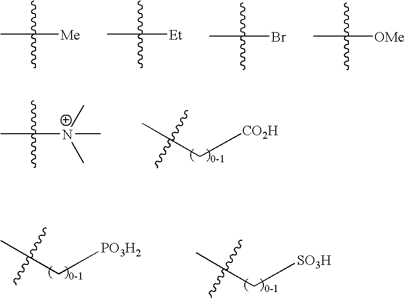

Preferred R₇ is selected from the following groups: hydrogen, oxygen, sulfur, amine, halide, hydroxyl (—OH), thiol (—SH), carbonyl, acidic, alkyl, alkenyl, carbocyclic, heterocyclic, substituted substitution group and combinations thereof. Particularly preferred R₇ is selected from the following groups: hydrogen, halide, thiol (—SH), carbonyl, acidic, alkyl, alkenyl, carbocyclic, substituted substitution group and combinations thereof. R₇ is preferably selected from the groups consisting of carbocyclic and substituted substitution group. The substituted substitution group for R₇ are especially preferred compounds or moieties such as:

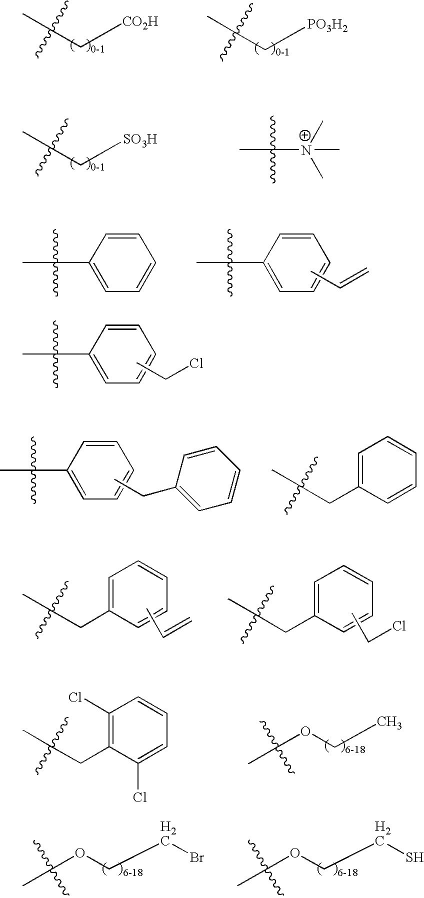

The aforementioned preferred selections for each substituent group R₁ through R₇ can be combined in each variation and permutation. In certain, preferred embodiments, for example, the inhibitor of the invention can comprise substituent groups wherein R₁ through R₇ are as follows: R₁ is preferably selected from the group consisting of alkyl, carbocyclic and substituted substitution group; R₂ is preferably selected from the group consisting of halide, alkyl and substituted substitution group; R₃ is preferably selected from the group consisting of carbonyl, acylamino, oximyl, hydrazyl, and substituted substitution group; R₄ and R₅ are each preferably independently selected from the group consisting of oxygen, hydroxyl (—OH), acidic, alkyl, and substituted substitution group; R₆ is preferably selected from the group consisting of amine, acidic, alkyl, and substituted substitution group; and R₇ is preferably selected from the groups consisting of carbocyclic and substituted substitution group.

Certain indole glyoxamides are particularly useful as PL A₂ inhibiting moieties in some embodiments. Specifically [2-(3-(2-amino-2-oxoacetyl)-1-(biphenyl-2-ylmethyl)-2-methyl-1H-indol-4-yloxy)acetic acid], shown in FIG. 2, alternatively referred to herein as ILY-4001 and/or as methyl indoxam has been found to be an effective phospholipase inhibitor or inhibiting moiety. This indole compound is represented by the structure below, as formula (V):

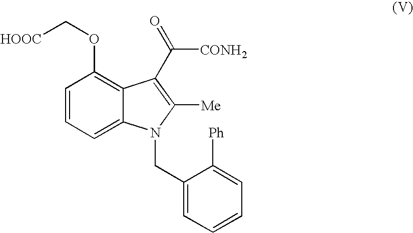

This compound has been shown, based on in-vitro assays, to have phospholipase activity for a number of PLA2 classes, and is a strong inhibitor of mouse and human PLA2IB enzymes in vitro (Singer, Ghomashchi et al. 2002; Smart, Pan et al. 2004). This indole compound was synthesized (See, Example 4) and as noted above, was evaluated in-vivo for phospholipase-A2 inhibition in a mice model. (See, Example 5, including Examples 5A through 5C). This indole compound was characterized with respect to inhibition activity, absorption and bioavailability. (See, Example 6, including Examples 6A through 6C).

Other indole compounds are also included within the scope of this invention. Many indoles have been described in the literature, for example, in connection with reported structure-activity-relationship studies (Schevitz, Bach et al. 1995; Dillard, Bach et al. 1996; Dillard, Bach et al. 1996; Draheim, Bach et al. 1996; Mihelich and Schevitz 1999). Table 1 lists various indole compounds, together with reported activity data against different phospholipase enzymes, including: human non-pancreatic PLA2 (hnp PLA2), human pancreatic secreted PLA2 (hps PLA2), and porcine pancreatic secreted PLA2 (pps PLA2).

TABLE 1
Indole Compounds

| | IC₅₀ | IC₅₀ | IC₅₀ |
|---|---|---|---|
| | (μM) | (μM) | (μM) |
| Structure | hnp PLA₂ | hps PLA₂ | pps PLA₂ |
| 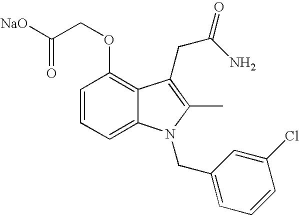 | 0.052 ± 0.012 | 1.2 | 0.02 |
| 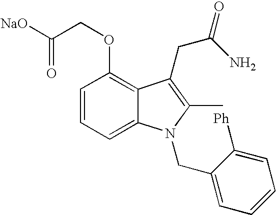 | 0.010 ± 0.001 | 4.09 | 0.014 |
| 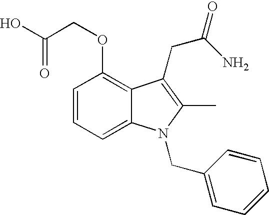 | 0.052 ± 0.010 | 1.4 | 0.15 |
| 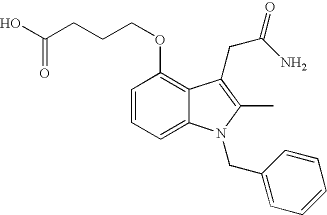 | 0.399 ± 0.045 | 3.66 | 0.61 |
| 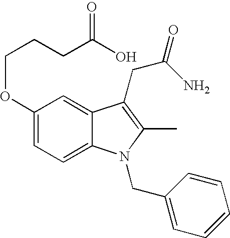 | 0.152 ± 0.033 | 69 | 25 |
| 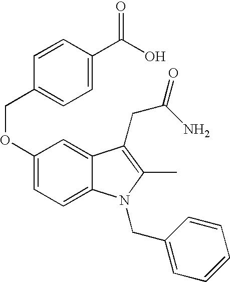 | 0.147 ± 0.009 | 22.5 | 7.5 |
| 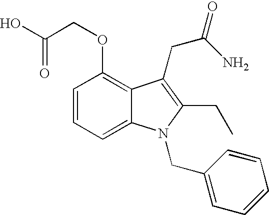 | 0.024 ± 0.001 | 1.8 | 0.13 |
| 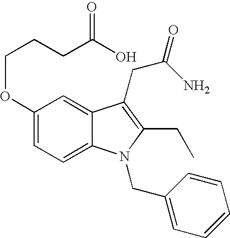 | 0.189 ± 0.006 | 94 | 13.5 |
| 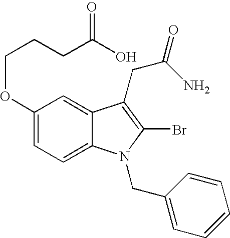 | 0.073 ± 0.016 | 15.9 | 2.86 |
| 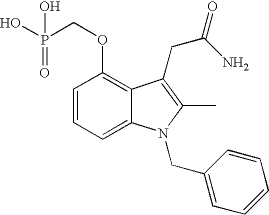 | 1.29 ± 0.16 | 73.5 | 5.55 |
| 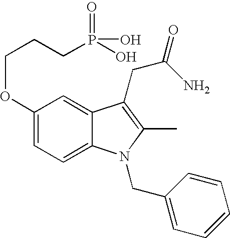 | 0.057 ± 0.004 | 67 | 27 |
| 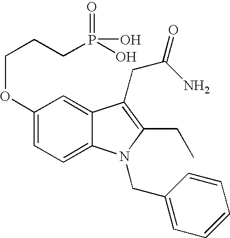 | 0.023 ± 0.005 | 91.1 | 35.5 |
| 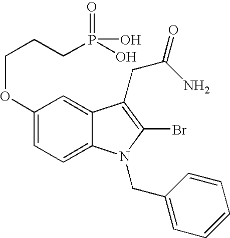 | 0.033 ± 0.004 | 6.2 | 2.2 |
| 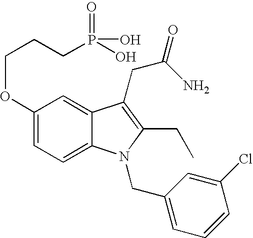 | 0.016 ± 0.010 | 46.2 | |
| 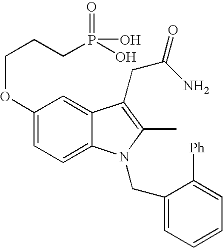 | 0.022 ± 0.006 | 39 | 7.6 |
| 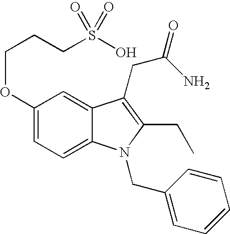 | 0.050 ± 0.015 | 135 | 5.8 |
| 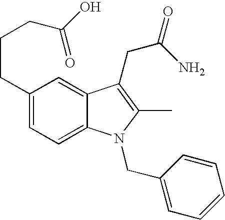 | 0.155 ± 0.029 | 94 | |
| 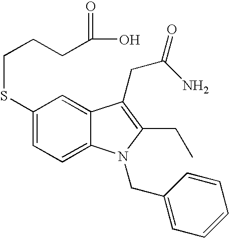 | 0.023 ± 0.005 | 16 | |
| 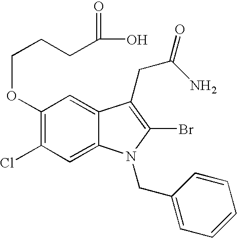 | 0.020 ± 0.003 | 3.2 | 1.3 |
| 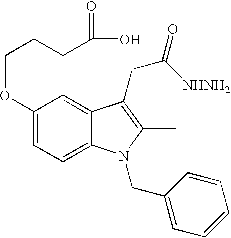 | 1.020 ± 0.150 | no activity | no activity |
| 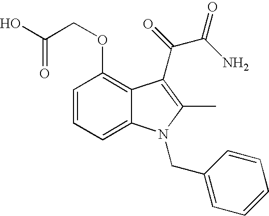 | 0.011 ± 0.004 | 0.761 | 0.015 |
| 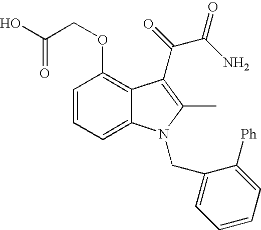 | 0.006 ± 0.001 | 0.364 | 0.097 |
| 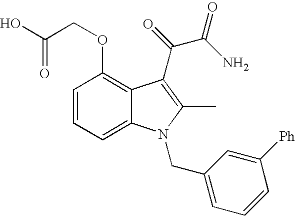 | 0.009 ± 0.001 | 0.57 | 0.007 |
| 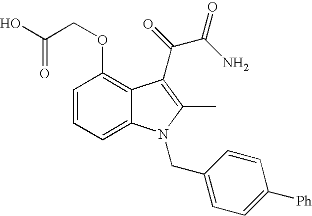 | 0.043 ± 0.003 | 1.09 | |
| 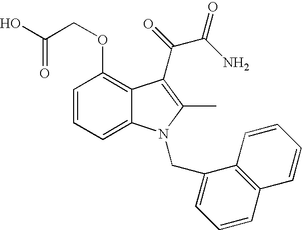 | 0.009 ± 0.004 | 1.2 | |
| 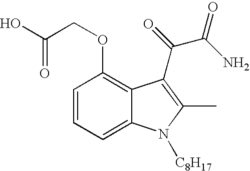 | 0.008 ± 0.003 | 0.78 | |
| 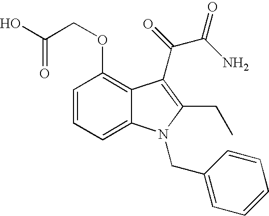 | 0.009 ± 0.001 | 0.228 | 0.048 |
| 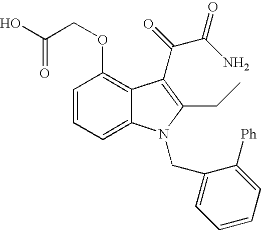 | 0.004 ± 0.001 | 0.062 | |
| 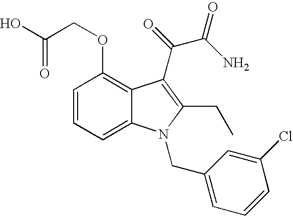 | 0.007 ± 0.002 | 0.39 | 0.003 |
| 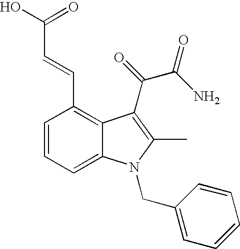 | 46 | >100 | |
| 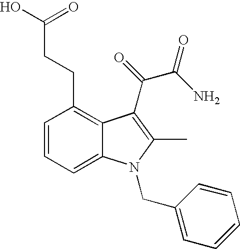 | 0.145 ± 0.006 | >100 | |
| 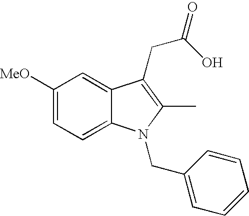 | 13.6 ± 4.2 | | |
| 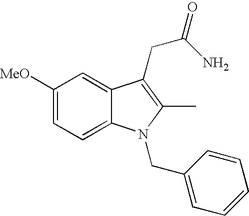 | 0.84 ± 0.17 | | |
| 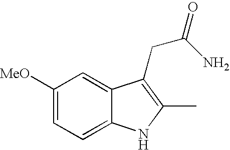 | | | |
| 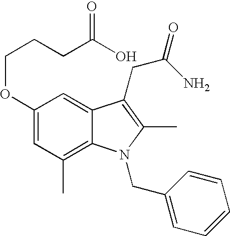 | 0.075 ± 0.013 | | |

Other indole compounds can be employed within the scope of this invention. Table 2 lists some of such other indole compounds.

TABLE 2
Indole Compounds
Indole glyoxamides

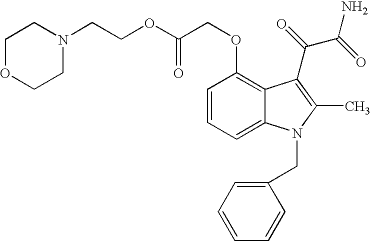

Indoly containing sulfonamides

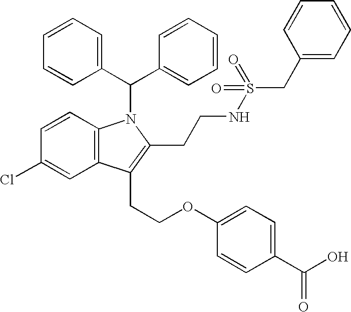

Other compounds having fused five-membered rings and six-membered rings with at least one heteroatom (referred to herein generally as indole-related compounds) can also be used in connection with the present invention. Table 3 lists some of such other indole-related compounds, and as relevant, patent references.

TABLE 3
Indole-Related Compounds

| Scaffolds | Structures | Patent # |
|---|---|---|
| Indole acetamide/ glyoxamides | 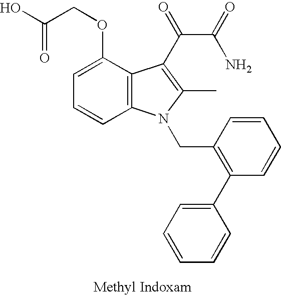 | WO9921559 |
| Indole glyoxamides | 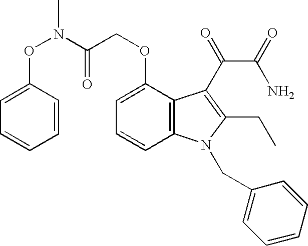 | WO0121587 |
| Benzothiophene | 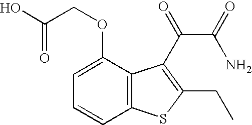 | |
| Indolizine | 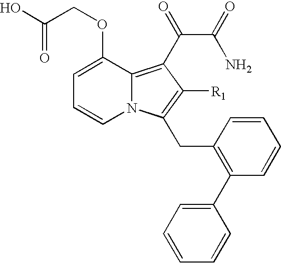 | US 6645976 |
| Indene | 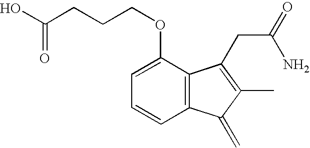 | US 6214876 |
| Substituted Tricyclic | 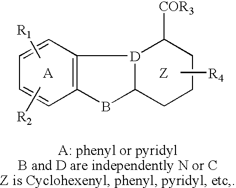 | WO9818464 |
| Bicyclic Pyrrole- Pyrimidine | 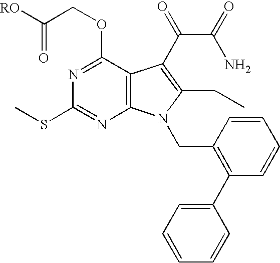 | |
| Carbazole | 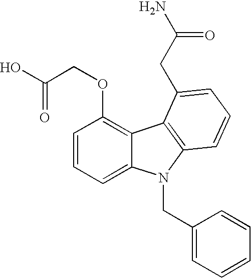 | WO03014082 |
| Cyclopenta-Indole | 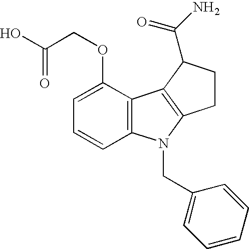 | |
| Cyclohepta-Indole | 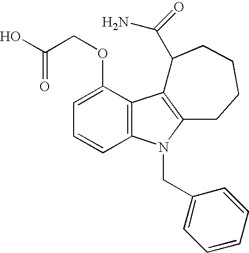 | WO03016277 |

With reference to FIGS. 6C and 6D, indole-compounds of the invention can generally include “inverse indole compounds” that are mirror-image analogues of the core structure of the corresponding indole based on a reference axis taken orthogonal to and bisecting the fused bond between the five-membered and six-membered ring core, but that maintain the defined substituent groups at the same position. (See FIG. 6C compared to FIG. 6D). Indole compounds and indole-related compounds of the invention can also include “reciprocal indole compounds” and “reciprocal indole-related compounds” that are mirror-image analogues of the core structure of the corresponding indole based on a reference axis taken along the axis of the fused bond between the five-membered and six-membered ring core, but which maintain at least each of the-R₃ and-R₄ positions and each of the-R₁ and-R₇ at the same position, and that maintain-R₂ and at least one of-R₅ and-R₆ at the same position.

The salts of all of the above-described indole-related compounds and above-described indole compounds, including those represented by formulae (I) through (V), are an additional aspect of the invention. In those instances where the compounds of the invention possess acidic or basic functional groups various salts may be formed which are more water soluble and physiologically suitable than the parent compound.

Representative pharmaceutically acceptable salts, include but are not limited to, the alkali and alkaline earth salts such as lithium, sodium, potassium, calcium, magnesium, aluminum and the like. Salts are conveniently prepared from the free acid by treating the acid in solution with a base or by exposing the acid to an ion exchange resin. Included within the definition of pharmaceutically acceptable salts are the relatively non-toxic, inorganic and organic base addition salts of compounds of the present invention, for example, ammonium, quaternary ammonium, and amine cations, derived from nitrogenous bases of sufficient basicity to form salts with the compounds of this invention (see, for example, S. M. Berge, et al., “Pharmaceutical Salts,” J. Phar. Sci., 66: 1-19 (1977)). Moreover, the basic group (s) of the compound of the invention may be reacted with suitable organic or inorganic acids to form salts such as acetate, benzenesulfonate, benzoate, bicarbonate, bisulfate, bitartrate, borate, bromide, camsylate, carbonate, chloride, clavulanate, citrate, chloride, edetate, edisylate, estolate, esylate, fluoride, fumarate, gluceptate, gluconate, glutamate, glycolylarsanilate, hexylresorcinate, bromide, chloride, hydroxynaphthoate, iodide, isothionate, lactate, lactobionate, laurate, malate, malseate, mandelate, mesylate, methylbromide, methylnitrate, methylsulfate, mucate, napsylate, nitrate, oleate, oxalate, palmitate, pantothenate, phosphate, polygalacturonate, salicylate, stearate, subacetate, succinate, tannate, tartrate, tosylate, trifluoroacetate, trifluoromethane sulfonate, and valerate.

Those of skill in the art will recognize that the compounds described herein may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the compounds having one or more of the utilities described herein, as well as mixtures of these various different forms. Prodrugs and active metabolites of the compounds described herein are also within the scope of the present invention.

Phospholipases and Inhibition Thereof Using Indoles and Indole-Related Compounds

Generally, in embodiments included within the various aspects of the invention, phospholipase inhibitors of the present invention can modulate or inhibit (e.g., blunt or reduce) the catalytic activity of phospholipases, preferably phospholipases secreted or contained in the gastrointestinal tract, including the gastric compartment, and more particularly the duodenum and/or the small intestine. For example, such enzymes preferably include, but are not limited to, secreted Group IB phospholipase A₂ (PL A₂-IB), also referred to as pancreatic phospholipase A₂ (p-PL A₂) and herein referred to as “PL A₂ IB” or “phospholipase-A₂ IB. Such enzymes can also include other phospholipase A2's secreted, such as Group IIA phospholipase A₂ (PL A₂ IIA). In some embodiments, particularly in connection with preferred indole compounds of the invention and preferred indole-related compounds of the invention, other phospholipases can also be considered within the scope of invention, including for example: phospholipase A1 (PLA₁); phospholipase B (PLB); phospholipase C (PLC); and phospholipase D (PLD). The inhibitors of the invention preferably inhibit the activity at least the phospholipase-A₂ IB enzyme.

In some embodiments, the inhibitors of the present invention are specific, or substantially specific for inhibiting phospholipase activity, such as phospholipase A₂ activity (including for example phospholipase-A₂ IB). For example, in some preferred embodiments inhibitors of the present invention do not inhibit or do not significantly inhibit or essentially do not inhibit lipases, such as pancreatic triglyceride lipase (PTL) and carboxyl ester lipase (CEL). In some preferred embodiments, inhibitors of the present invention inhibit PL A₂, and preferably phospholipase-A₂ IB, but in each case do not inhibit or do not significantly inhibit or essentially do not inhibit any other phospholipases; in some preferred embodiments, inhibitors of the present invention inhibit PL A₂, and preferably phospholipase-A₂ IB, but in each case do not inhibit or do not significantly inhibit or essentially do not inhibit PLA₁; in some preferred embodiments, inhibitors of the present invention inhibit PL A₂, and preferably phospholipase-A₂ IB, but do not inhibit or do not significantly inhibit or essentially do not inhibit PLB. In some embodiments, the phospholipase inhibitor does not act on the gastrointestinal mucosa, for example, it does not inhibit or does not significantly inhibit or essentially does not inhibit membrane-bound phospholipases.

The different activities of PL A₂, PL A₁, and PLB are generally well-characterized and understood in the art. PL A₂ hydrolyzes phospholipids at the sn-2 position liberating 1-acyl lysophospholipids and fatty acids; PL A₁ acts on phospholipids at the sn-1 position to release 2-acyl lysophospholipids and fatty acids; and phospholipase B cleaves phospholipids at both sn-1 and sn-2 positions to form a glycerol and two fatty acids. See, e.g., Devlin, Editor, Textbook of Biochemistry with Clinical Correlations, 5^{th} ed. Pp 1104-1110 (2002).

Phospholipids substrates acted upon by gastrointestinal PL A₁, PL A₂ (including phospholipase-A₂ IB) and PLB are mostly of the phosphatidylcholine and phosphatidylethanolamine types, and can be of dietary or biliary origin, or may be derived from being sloughed off of cell membranes. For example, in the case of phosphatidylcholine digestion, PL A₁ acts at the sn-1 position to produce 2-acyl lysophosphatidylcholine and free fatty acid; PL A₂ acts at the sn-2 position to produce 1-acyl lysophosphatidylcholine and free fatty acid; while PLB acts at both positions to produce glycerol 3-phosphorylcholine and two free fatty acids (Devlin, 2002).

Pancreatic PL A₂ (and phospholipase-A₂ IB) is secreted by acinar cells of the exocrine pancreas for release in the duodenum via pancreatic juice. PL A₂ (and phospholipase-A₂ IB) is secreted as a proenzyme, carrying a polypeptide chain that is subsequently cleaved by proteases to activate the enzyme's catalytic site. Documented structure-activity-relationships (SAR) for PL A₂ isozymes illustrate a number of common features (see for instance, Gelb M., Chemical Reviews, 2001, 101:2613-2653; Homan, R., Advances in Pharmacology, 1995, 12:31-66; and Jain, M. K., Intestinal Lipid Metabolism, Biology, pathology, and interfacial enzymology of pancreatic phospholipase A₂, 2001, 81-104, each incorporated herein by reference).

The inhibitors of the present invention can take advantage of certain of these common features to inhibit phospholipase activity and especially PL A₂ activity. Common features of PL A₂ enzymes include sizes of about 13 to about 15 kDa; stability to heat; and 6 to 8 disulfides bridges. Common features of PL A₂ enzymes also include conserved active site architecture and calcium-dependent activities, as well as a catalytic mechanism involving concerted binding of His and Asp residues to water molecules and a calcium cation, in a His-calcium-Asp triad. A phospholipid substrate can access the catalytic site by its polar head group through a slot enveloped by hydrophobic and cationic residues (including lysine and arginine residues) described in more detail below. Within the catalytic site, the multi-coordinated calcium ion activates the acyl carbonyl group of the sn-2 position of the phospholipid substrate to bring about hydrolysis (Devlin, 2002). In some preferred embodiments, inhibitors of the present invention inhibit this catalytic activity of PL A₂ by interacting with its catalytic site.

PL A₂ enzymes are active for catabolizing phospholipids substrates primarily at the lipid-water interface of lipid aggregates found in the gastrointestinal lumen, including, for example, fat globules, emulsion droplets, vesicles, mixed micelles, and/or disks, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites. Such enzymes can be considered to act while “docked” to a lipid-water interface. In such lipid aggregates, the phospholipid substrates are typically arranged in a mono layer or in a bilayer, together with one or more other components listed above, which form part of the outer surface of the aggregate. The surface of a phospholipase bearing the catalytic site contacts this interface facilitating access to phospholipid substrates. This surface of the phospholipase is known as the i-face, i.e., the interfacial recognition face of the enzyme. The structural features of the i-face of PL A₂ have been well documented. See, e.g., Jain, M. K, et al, Methods in Enzymology, vol. 239, 1995, 568-614, incorporated herein by reference. The inhibitors of the present invention can take advantage of these structural features to inhibit PL A₂ activity. For instance, it is known that the aperture of the slot forming the catalytic site is normal to the i-face plane. The aperture is surrounded by a first crown of hydrophobic residues (mainly leucine and isoleucine residues), which itself is contained in a ring of cationic residues (including lysine and arginine residues).

As noted, PL A₂ enzymes share a conserved active site architecture and a catalytic mechanism involving concerted binding of His and Asp residues to water molecules and a calcium cation. Without being bound by theory, a phospholipid substrate can access the catalytic site of such enzymes with its polar head group directed through a slot enveloped by hydrophobic and cationic residues. Within the catalytic site, the multi-coordinated calcium ion activates the acyl carbonyl group of the sn-2 position of the phospholipid substrate to bring about hydrolysis.

In view of the substantial structure-activity-relationship studies for phospholipase-A2 enzymes, considered together with the significant experimental data demonstrated in Example 5 (including Examples 5A through 5C), a skilled person can appreciate that the observed inhibitive effect of ILY-4001 can be realized in other indole compounds of the invention (having the identical core structure) as well as in indole-related compounds comprising a fused five-membered ring and six-membered ring. In particular, without being bound by theory not expressly recited in the claims, a skilled person can appreciate, with reference to FIG. 6A, for example, that substituents at positions 3 and 4 and 5 of the indole structure can be selected and evaluated to be effective for polar interaction with the enzyme and with calcium ion (associated with the calcium-dependent phospholipase activity). Similarly, a person of skill in the art can appreciate that the substituents at positions 1 and 2 of the indole structure can be selected and evaluated to be relatively hydrophobic. Considered in combination, the polar groups at positions 3, 4 and 5 and the relatively hydrophobic groups at positions 1 and 2 can effectively associate the inhibitor (or inhibiting moiety) with a hydrophilic lipid-water interface (via the hydrophobic regions), and also orient the inhibitor (or inhibiting moiety) such that its polar region can be effectively positioned into the enzyme pocket—with its polar head group directed through a slot enveloped by hydrophobic and cationic residues. Similarly, with reference to FIG. 6B, for example, one can appreciate that corresponding groups on the indole-related compound shown therein can have the same functionality. Specifically, a person of skill in the art can appreciate that substituents at positions R₃, R₄ and R₅ of the indole-related structure can be selected and evaluated to be effective for polar interaction with the enzyme and with calcium ion, and that the substituents at positions R₁ and R₂ of the indole-related structure can be selected and evaluated to be relatively hydrophobic.

Similarly, with reference to FIGS. 6C and 6D, the above-described inverse indole compounds that are mirror-image analogues of the core structure of the corresponding indole of interest, and the above-described reciprocal indole compounds and reciprocal indole-related compounds that are alternative mirror-image analogues of the core structure of the corresponding indole or related compound can be similarly configured with polar substituents and hydrophobic substituents to provide alternative indole structures and alternative indole-related structures within the scope of the invention.

Moreover, a person skilled in the art can evaluate particular inhibitors within the scope of this invention using known assaying and evaluation approaches. For example, the extent of inhibition of the inhibitors of the invention can be evaluated using in-vitro assays (See, for example, Example 6A) and/or in-vivo studies (See, for example, Example 5). Further, binding of a phospholipase inhibitor to a phospholipase enzyme can be evaluated by nuclear magnetic resonance, for example to provide identification of sites essential or non-essential for such binding interaction. Additionally, one of skill in the art can use available structure-activity relationship (SAR) for phospholipase inhibitors that suggest positions where structural variations are allowed. A library of candidate phospholipase inhibitors can be designed to feature different points of attachment of the phospholipase inhibiting moiety, e.g., chosen based on information described above as well as randomly, so as to present the phospholipase inhibiting moiety in multiple distinct orientations. Candidates can be evaluated for phospholipase inhibiting activity to obtain phospholipase inhibitors with suitable attachment points of the phospholipase inhibiting moiety to the polymer moiety or other non-absorbed moiety.

Generally, the extent of inhibition is not narrowly critical to the invention, but can be of significance in particular embodiments. Hence, the term “inhibits” and its grammatical variations are not intended to require a complete inhibition of enzymatic activity. For example, it can refer to a reduction in enzymatic activity by at least about 50%, at least about 75%, preferably by at least about 90%, more preferably at least about 98%, and even more preferably at least about 99% of the activity of the enzyme in the absence of the inhibitor. Most preferably, it refers to a reduction in enzyme activity by an effective amount that is by an amount sufficient to produce a therapeutic and/or a prophylactic benefit in at least one condition being treated. in a subject receiving phospholipase inhibiting treatment, e.g., as disclosed herein. Conversely, the phrase “does not inhibit” and its grammatical variations does not require a complete lack of effect on the enzymatic activity. For example, it refers to situations where there is less than about 20%, less than about 10%, less than about 5%, preferably less than about 2%, and more preferably less than about 1% of reduction in enzyme activity in the presence of the inhibitor. Most preferably, it refers to a minimal reduction in enzyme activity such that a noticeable effect is not observed. Further, the phrase “does not significantly inhibit” and its grammatical variations refers to situations where there is less than about 40%, less than about 30%, less than about 25%, preferably less than about 20%, and more preferably less than about 15% of reduction in enzyme activity in the presence of the inhibitor. Further, the phrase “essentially does not inhibit” and its grammatical variations refers to situations where there is less than about 30%, less than about 25%, less than about 20%, preferably less than about 15%, and more preferably less than about 10% of reduction in enzyme activity in the presence of the inhibitor.

The inhibitors can modulate phospholipase activity by reversible and/or irreversible inhibition. Reversible inhibition by a phospholipase inhibitor of the present invention may be competitive (e.g. where the inhibitor binds to the catalytic site of a phospholipase), noncompetitive (e.g., where the inhibitor binds to an allosteric site of a phospholipase to effect an allosteric change), and/or uncompetitive (where the inhibitor binds to a complex between a phospholipase and its substrate). Inhibition may also be irreversible, where the phospholipase inhibitor remains bound, or significantly remains bound, or essentially remains bound to a site on a phospholipase without dissociating, without significantly dissociating, or essentially without dissociating from the enzyme.

Lumen-Localized PLA2-Inhibitors

As noted above, in some embodiments, the PLA2 inhibitors of the invention are preferably lumen-localized PLA2 inhibitors. Such phospholipase inhibitors can be adapted for having both lumen-localization functionality as well as enzyme-inhibition functionalization. In some schema, certain aspects of such dual functionality can be achieved synergistically (e.g., by using the same structural features and/or charge features); in other schema, the lumen-localization functionality can be achieved independently (e.g., using different structural and/or charge features) from the enzyme-inhibition functionality.

The compound 2-(3-(2-amino-2-oxoacetyl)-1-(biphenyl-2-ylmethyl)-2-methyl-1H-indol-4-yloxy)acetic acid, shown in FIG. 2, and referred to herein as ILY-4001 (or methyl indoxam) was evaluated to consider its absorption using in-vitro Caco-2 cell assays (See Example 6B) and using bioavailability in in-vivo studies (See, for example, Example 6C). Bioavailability of this compound can be reduced, and reciprocally, lumen-localization can be improved, according to this preferred embodiment of the invention, for example, by charge modification and/or by covalently linking this indole moiety to a polymer. (See, for example, co-owned PCT Application No. US/2005/______ entitled “Phospholipase Inhibitors Localized in the Gastrointestinal Lumen” filed on May 3, 2005 by Charmot et al.), incorporated herein by reference.

The phospholipase inhibitors of the invention are preferably localized in the gastrointestinal lumen, such that upon administration to a subject, the phospholipase inhibitors remain substantially in the gastrointestinal lumen. Following administration, the localized phospholipase inhibitors can remain in and pass naturally through the gastrointestinal tract, including the stomach, the duodenum, the small intestine and the large intestine (until passed out of the body via the gastrointestinal tract). The phospholipase inhibitors are preferably substantially stable (e.g., with respect to composition and/or with respect to functionality for inhibiting phospholipase) while passing through at least the stomach and the duodenum, and more preferably, are substantially stable while passing through the stomach, the duodenum and the small intestine of the gastrointestinal tract, and most preferably, are substantially stable while passing through the entire gastrointestinal tract. The phospholipase inhibitors can act in the gastrointestinal lumen, for example to catabolize phospholipase substrates or to modulate the absorption and/or downstream activities of products of phospholipase digestion.

Phospholipase inhibitors are localized within the gastrointestinal lumen, in one approach, by being not absorbed through a gastrointestinal mucosa. As another approach, the phospholipase inhibitors can be localized in the gastrointestinal lumen by being absorbed into a mucosal cell and then effluxed back into a gastrointestinal lumen.

Generally, without being constrained by categorization into one or more of the aforementioned general approaches by which the phospholipase inhibitor can be lumen-localized, preferred phospholipase inhibitors of the invention (as contemplated in the various aspects of the invention) can be realized by several general lumen-localization embodiments. In one general lumen-localization embodiment, for example, the phospholipase inhibitor can comprise an oligomer or polymer moiety covalently linked, directly or indirectly through a linking moiety, to a phospholipase inhibiting moiety of the invention—including the afore-described indole-related compounds and indole-compounds described herein. In a further general embodiment, the lumen-localized phospholipase inhibitor can be a substituted small organic molecule itself-including the indole-related compounds and indole-compounds described above.

In general for each various aspects and embodiments included within the various aspects of the invention, the inhibitor can be localized, upon administration to a subject, in the gastrointestinal lumen of the subject, such as an animal, and preferably a mammal, including for example a human as well as other mammals (e.g., mice, rats, rabbits, guinea pigs, hamsters, cats, dogs, porcine, poultry, bovine and horses). The term “gastrointestinal lumen” is used interchangeably herein with the term “lumen,” to refer to the space or cavity within a gastrointestinal tract, which can also be referred to as the gut of the animal. In some embodiments, the phospholipase inhibitor is not absorbed through a gastrointestinal mucosa. “Gastrointestinal mucosa” refers to the layer(s) of cells separating the gastrointestinal lumen from the rest of the body and includes gastric and intestinal mucosa, such as the mucosa of the small intestine. In some embodiments, lumen localization is achieved by efflux into the gastrointestinal lumen upon uptake of the inhibitor by a gastrointestinal mucosal cell. A “gastrointestinal mucosal cell” as used herein refers to any cell of the gastrointestinal mucosa, including, for example, an epithelial cell of the gut, such as an intestinal enterocyte, a colonic enterocyte, an apical enterocyte, and the like. Such efflux achieves a net effect of non-absorbedness, as the terms, related terms and grammatical variations, are used herein.

In preferred approaches, the phosphate inhibitor can be an inhibitor that is substantially not absorbed from the gastrointestinal lumen into gastrointestinal mucosal cells. As such, “not absorbed” as used herein can refer to inhibitors adapted such that a significant amount, preferably a statistically significant amount, more preferably essentially all of the phospholipase inhibitor, remains in the gastrointestinal lumen. For example, at least about 80% of phospholipase inhibitor remains in the gastrointestinal lumen, at least about 85% of phospholipase inhibitor remains in the gastrointestinal lumen, at least about 90% of phospholipase inhibitor remains in the gastrointestinal lumen, at least about 95%, at least about 98%, preferably at least about 99%, and more preferably at least about 99.5% remains in the gastrointestinal lumen. Reciprocally, stated in terms of serum bioavailability, a physiologically insignificant amount of the phospholipase inhibitor is absorbed into the blood serum of the subject following administration to a subject. For example, upon administration of the phospholipase inhibitor to a subject, not more than about 20% of the administered amount of phospholipase inhibitor is in the serum of the subject (e.g., based on detectable serum bioavailability following administration), preferably not more than about 15% of phospholipase inhibitor, and most preferably not more than about 10% of phospholipase inhibitor is in the serum of the subject. In some embodiments, not more than about 5%, not more than about 2%, preferably not more than about 1%, and more preferably not more than about 0.5% is in the serum of the subject. In some cases, localization to the gastrointestinal lumen can refer to reducing net movement across a gastrointestinal mucosa, for example, by way of both transcellular and paracellular transport, as well as by active and/or passive transport. The phospholipase inhibitor in such embodiments is hindered from net permeation of a gastrointestinal mucosal cell in transcellular transport, for example, through an apical cell of the small intestine; the phospholipase inhibitor in these embodiments is also hindered from net permeation through the “tight junctions” in paracellular transport between gastrointestinal mucosal cells lining the lumen. The term “not absorbed” is used interchangeably herein with the terms “non-absorbed,” “non-absorbedness,” “non-absorption” and its other grammatical variations.

In some embodiments, an inhibitor or inhibiting moiety can be adapted to be non-absorbed by modifying the charge and/or size, particularly, as well as additionally other physical or chemical parameters of the phospholipase inhibitor. For example, in some embodiments, the phospholipase inhibitor is constructed to have a molecular structure that minimizes or nullifies absorption through a gastrointestinal mucosa. The absorption character of a drug can be selected by applying principles of pharmacodynamics, for example, by applying Lipinsky's rule, also known as “the rule of five.” As a set of guidelines, Lipinsky shows that small molecule drugs with (i) molecular weight, (ii) number of hydrogen bond donors, (iii) number of hydrogen bond acceptors, and (iv) water/octanol partition coefficient (Moriguchi logP) each greater than a certain threshold value generally do not show significant systemic concentration. See Lipinsky et al, Advanced Drug Delivery Reviews, 46, 2001 3-26, incorporated herein by reference. Accordingly, non-absorbed phospholipase inhibitors can be constructed to have molecule structures exceeding one or more of Lipinsky's threshold values, and preferably two or more, or three or more, or four or more or each of Lipinsky's threshold values. See also Lipinski et al., Experimental and computational approaches to estimate solubility and permeability in drug discovery and development settings, Adv. Drug Delivery Reviews, 46:3-26 (2001); and Lipinski, Drug-like properties and the causes of poor solubility and poor permeability, J. Pharm. & Toxicol. Methods, 44:235-249 (2000), incorporated herein by reference. In some preferred embodiments, for example, a phospholipase inhibitor of the present invention can be constructed to feature one or more of the following characteristics: (i) having a MW greater than about 500 Da; (ii) having a total number of NH and/or OH and/or other potential hydrogen bond donors greater than about 5; (iii) having a total number of O atoms and/or N atoms and/or other potential hydrogen bond acceptors greater than about 10; and/or (iv) having a Moriguchi partition coefficient greater than about 10⁵, i.e., log P greater than about 5. Any art known phospholipase inhibitors and/or any phospholipase inhibiting moieties described below can be used in constructing a non-absorbed molecular structure.

Preferably, the permeability properties of the compounds are screened experimentally: permeability coefficient can be determined by methods known to those of skill in the art, including for example by Caco-2 cell permeability assay. The human colon adenocarcinoma cell line, Caco-2, can be used to model intestinal drug absorption and to rank compounds based on their permeability. It has been shown, for example, that the apparent permeability values measured in Caco-2 monolayers in the range of 1×10⁻⁷ cm/sec or less typically correlate with poor human absorption (Artursson P, K. J. (1991). Permeability can also be determined using an artificial membrane as a model of a gastrointestinal mucosa. For example, a synthetic membrane can be impregnated with e.g. lecithin and/or dodecane to mimic the net permeability characteristics of a gastrointestinal mucosa. The membrane can be used to separate a compartment containing the phospholipase inhibitor from a compartment where the rate of permeation will be monitored. “Correlation between oral drug absorption in humans and apparent drug.” Biochemical and Biophysical Research Communications 175(3): 880-885.) Also, parallel artificial membrane permeability assays (PAMPA) can be performed. Such in vitro measurements can reasonably indicate actual permeability in vivo. See, for example, Wohnsland et al. J. Med. Chem., 2001, 44:923-930; Schmidt et al., Millipore corp. Application note, 2002, n^{o} AN1725EN00, and n^{o} AN1728EN00, incorporated herein by reference. The permeability coefficient is reported as its decimal logarithm, Log Pe.

In some embodiments, the phospholipase inhibitor permeability coefficient Log Pe is preferably lower than about −4, or lower than about −4.5, or lower than about −5, more preferably lower than about −5.5, and even more preferably lower than about −6 when measured in the permeability experiment described in Wohnsland et al. J. Med. Chem. 2001, 44. 923-930.

As noted, in one general lumen-localization embodiment, a phospholipase inhibitor can comprise a phospholipase inhibiting moiety such as the indole-related compounds and indole compounds described above, that are linked, coupled or otherwise attached to a non-absorbed oligomer or polymer moiety, where such oligomer or polymer moiety can be a hydrophobic moiety, hydrophilic moiety, and/or charged moiety. In some preferred embodiments, the phospholipase inhibiting moiety is coupled to a polymer moiety. Generally, such polymer inhibitor can be sized to be non-absorbed, and can be adapted to be enzyme-inhibiting, for example based on one or more or a combination of features, such as charge characteristics, relative balance and/or distribution of hydrophilic/hydrophobic character, and molecular structure. The oligomer or polymer in this general embodiment is preferably soluble, and can preferably be a copolymer (including polymers having two monomer-repeat-units, terpolymers and higher-order polymers), including for example random copolymer or block copolymer. The oligomer or polymer can generally include one or more ionic monomer moieties such as one or more anionic monomer moieties. The oligomer or polymer can generally include one or more hydrophobic monomer moieties.

In one more specific approach within this general embodiment, the polymer moiety may be of relatively high molecular weight, for example ranging from about 1000 Da to about 500,000 Da, preferably in the range of about 5000 to about 200,000 Da, and more preferably sufficiently high to hinder or preclude (net) absorption through a gastrointestinal mucosa. Large polymer moieties may be advantageous, for example, in scavenging approaches involving relatively large, soluble or insoluble (e.g., cross-linked) polymers having multiple inhibiting moieties (e.g., as discussed below in connection with FIG. 2).

In an alternative more specific approach within this general embodiment, the oligomer or polymer moiety may be of low molecular weight, for example not more than about 5000 Da, and preferably not more than about 3000 Da and in some cases not more than about 1000 Da. Preferably within this approach, the oligomer or polymer moiety can consist essentially of or can comprise a block of hydrophobic polymer, allowing the inhibitor to associate with a water-lipid interface.

### BIBLIOGRAPHY

The following references describe knowledge known in the art that relates to the present invention, for example, as indicated above. In some cases, these references are cited above in the description of the invention by reference to the first two authors and the year. These references are incorporated by reference herein.
Baker, R. R. and H. Chang (2000). “A metabolic path for the degradation of lysophosphatidic acid, an inhibitor of lysophosphatidylcholine lysophospholipase, in neuronal nuclei of cerebral cortex.” Biochim Biophys Acta 1483(1): 58-68.
Baker, R. R. and H. Y. Chang (1999). “Evidence for two distinct lysophospholipase activities that degrade lysophosphatidylcholine and lysophosphatidic acid in neuronal nuclei of cerebral cortex.” Biochim Biophys Acta 1438(2): 253-63.
Carriere (1993). “Secretion and contribution to Lipolysis of Gastic and Pancreatic Lipases During a Test Meal in Humans.” Gastroenterology: 876-888.
Carriere, F., C. Renou, et al. (2000). “The specific activities of human digestive lipases measured from the in vivo and in vitro lipolysis of test meals.” Gastroenterology 119(4): 949-60.
Duan, R. D. and B. Borgstrom (1993). “Is there a specific lysophospholipase in human pancreatic juice?” Biochim Biophys Acta 1167(3): 326-30.
Dunlop, M. E., E. Muggli, et al. (1997). “Differential disposition of lysophosphatidylcholine in diabetes compared with raised glucose: implications for prostaglandin production in the diabetic kidney glomerulus in vivo.” Biochim Biophys Acta 1345(3): 306-16.
el Soda, M., L. Pannell, et al. (1989). “Microencapsulated enzyme systems for the acceleration of cheese ripening.” J Microencapsul 6(3): 319-26.
Flieger, A., S. Gong, et al. (2001). “Novel lysophospholipase A secreted by Legionella pneumophila.” J Bacteriol 183(6): 2121-4.
Flieger, A., B. Neumeister, et al. (2002). “Characterization of the gene encoding the major secreted lysophospholipase A of Legionella pneumophila and its role in detoxification of lysophosphatidylcholine.” Infect Immun 70(11): 6094-106.
Gesta, S., M. F. Simon, et al. (2002). “Secretion of a lysophospholipase D activity by adipocytes: involvement in lysophosphatidic acid synthesis.” J Lipid Res 43(6): 904-10.
McMorn, P. and G. J. Hutchings (2004). “Heterogeneous enantioselective catalysts: strategies for the immobilisation of homogeneous catalysts.” Chem Soc Rev 33(2): 108-22.
Millan, C. G., M. L. Marinero, et al. (2004). “Drug, enzyme and peptide delivery using erythrocytes as carriers.” J Control Release 95(1): 27-49.
Muzykantov, V. R. (2001). “Delivery of antioxidant enzyme proteins to the lung.” Antioxid Redox Signal 3(1): 39-62.
Ross, B. M. and S. J. Kish (1994). “Characterization of lysophospholipid metabolizing enzymes in human brain.” J Neurochem 63(5): 1839-48.
Sakagami, H., J. Aoki, et al. (2005). “Biochemical and molecular characterization of a novel choline-specific glycerophosphodiester phosphodiesterase belonging to the nucleotide pyrophosphatase/phosphodiesterase (NPP) family.” J Biol. Chem.
Shah, N. P. (2000). “Probiotic bacteria: selective enumeration and survival in dairy foods.” J Dairy Sci 83(4): 894-907.
Shanado, Y., M. Kometani, et al. (2004). “Lysophospholipase I identified as a ghrelin deacylation enzyme in rat stomach.” Biochem Biophys Res Commun 325(4): 1487-94.
Sunaga, H., H. Sugimoto, et al. (1995). “Purification and properties of lysophospholipase isoenzymes from pig gastric mucosa.” Biochem J 308 (Pt 2): 551-7.
Taniyama, Y., S. Shibata, et al. (1999). “Cloning and expression of a novel lysophospholipase which structurally resembles lecithin cholesterol acyltransferase.” Biochem Biophys Res Commun 257(1): 50-6.
Tokumura, A., Y. Kanaya, et al. (2002). “Increased formation of lysophosphatidic acids by lysophospholipase D in serum of hypercholesterolemic rabbits.” J Lipid Res 43(2): 307-15.
Tokumura, A., E. Majima, et al. (2002). “Identification of human plasma lysophospholipase D, a lysophosphatidic acid-producing enzyme, as autotaxin, a multifunctional phosphodiesterase.” J Biol Chem 277(42): 39436-42.
Tosti, E., L. Dahl, et al. (1999). “Endothelial degradation of extracellular lysophosphatidylcholine.” Scand J Clin Lab Invest 59(4): 249-57.
Toyoda, T., H. Sugimoto, et al. (1999). “Sequence, expression in Escherichia coli, and characterization of lysophospholipase II.” Biochim Biophys Acta 1437(2): 182-93.
Walde, P. and S. Ichikawa (2001). “Enzymes inside lipid vesicles: preparation, reactivity and applications.” Biomol Eng 18(4): 143-77.
Wang, A. and E. A. Dennis (1999). “Mammalian lysophospholipases.” Biochim Biophys Acta 1439(1): 1-16.
Wang, A., H. C. Yang, et al. (1999). “A specific human lysophospholipase: cDNA cloning, tissue distribution and kinetic characterization.” Biochim Biophys Acta 1437(2): 157-69.
Witt, W., A. Mertsching, et al. (1984). “Secretion of phospholipase B from Saccharomyces cerevisiae.” Biochim Biophys Acta 795(1): 117-24.
Witt, W., M. E. Schweingruber, et al. (1984). “Phospholipase B from the plasma membrane of Saccharomyces cerevisiae. Separation of two forms with different carbohydrate content.” Biochim Biophys Acta 795(1): 108-16.
Wright, L. C., J. Payne, et al. (2004). “Cryptococcal phospholipases: a novel lysophospholipase discovered in the pathogenic fungus Cryptococcus gattii.” Biochem J 384(Pt 2): 377-84.

### EXAMPLES

### Example 1

### Reduction in Insulin Resistance in a Mouse Model

A phospholipase inhibitor, for example a composition comprising a phospholipase inhibiting moiety disclosed herein, can be used in a mouse model to demonstrate, for example, suppression of diet-induced insulin resistance, relating to, for example, diet-induced onset of diabetes. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

The duration of the study is typically about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight.

At the end of the about 6 to about 8 week study period, an oral glucose tolerance test and/or an insulin sensitivity test can be conducted as follows:

Oral glucose tolerance test—after an overnight fast, mice from each dosing group can be fed a glucose bolus (e.g., by stomach gavage using about 2 g/kg body weight) in about 50 μl of saline. Blood samples can be obtained from the tail vein before, and about 15, about 30, about 60, and about 120 minutes after glucose administration; blood glucose levels at the various time points can then be determined.

Insulin sensitivity test—after about a 6 hour morning fast, mice in each of the dosing groups can be administered bovine insulin (e.g., about 1 U/kg body weight, using, e.g., intraperitoneal administration. Blood samples can be obtained from the tail vein before, and about 15, about 30, about 60, and about 120 minutes after insulin administration; plasma insulin levels at the various time points can then be determined, e.g., by radioimmunoassay.

The effect of the non-absorbed phospholipase inhibitor, e.g., a phospholipase A2 inhibitor, is a decrease in insulin resistance, i.e., better tolerance to glucose challenge by, for example, increasing the efficiency of glucose metabolism in cells, and in the animals of the dose-treated groups fed a Western (high fat/high cholesterol) diet relative to the animals of the control groups. Effective dosages can also be determined.

### Example 2

### Reduction in Fat Absorption in a Mouse Model

A phospholipase inhibitor, for example a composition comprising a phospholipase inhibiting moiety disclosed herein, can be used in a mouse model to demonstrate, for example, reduced lipid absorption in subjects on a Western diet. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to 90 mg/kg body weight.

The mouse chow used may have a composition representative of a Western-type (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Triglyceride measurements can be taken for a duration of about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight. On a weekly basis, plasma samples can be obtained from the subject animals and analyzed for total triglycerides, for example, to determine the amount of lipids absorbed into the blood circulation.

The effect of the non-absorbed phospholipase inhibitor, e.g., a phospholipase A2 inhibitor, is a net decrease in lipid plasma levels, which indicates reduced fat absorption, in the animals of the dose-treated groups fed a Western (high fat/high cholesterol) diet relative to the animals of the control groups. Effective dosages can also be determined.

### Example 3

### Reduction in Diet-Induced Hypercholesterolemia in a Mouse Model

A phospholipase inhibitor, for example a composition comprising a phospholipase inhibiting moiety disclosed herein, can be used in a mouse model to demonstrate, for example, suppression of diet-induced hypercholesterolemia. The phospholipase inhibitor can be administered to subject animals either as a chow supplement and/or by oral gavage BID (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western-type (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Cholesterol and/or triglyceride measurements can be taken for a duration of about 6 to about 8 weeks, with the subject animals being dosed every day during this period. Typical dosing groups, containing about 6 to about 8 animals per group, can be composed of a untreated control group, a vehicle control group, and dose-treated groups ranging from about 10 mg/kg body weight to about 90 mg/kg body weight. On a weekly basis, plasma samples can be obtained from the subject animals and analyzed for total cholesterol and/or triglycerides, for example, to determine the amount of cholesterol and/or lipids absorbed into the blood circulation. Since most plasma cholesterol in a mouse is associated with HDL (in contrast to the LDL association of most cholesterol in humans), HDL and non-HDL fractions can be separated to aid determination of the effectiveness of the non-absorbed phospholipase inhibitor in lowering plasma non-HDL levels, for example VLDL/LDL.

The effect of the non-absorbed phospholipase inhibitor, e.g., a phospholipase A2 inhibitor, is a net decrease in hypercholesterolemia in the animals of the dose-treated groups fed a Western (high fat/high cholesterol) diet relative to the animals of the control groups. Effective dosages can also be determined.

### Example 4

### Synthesis of ILY-4001 [2-(3-(2-amino-2-oxoacetyl)-1-(biphenyl-2-ylmethyl)-2-methyl-1H-indol-4-yloxy)acetic acid] (Me Indoxam)

This example synthesized a compound for use as a phospholipase inhibitor or inhibiting moiety. Specifically, the compound 2-(3-(2-amino-2-oxoacetyl)-1-(biphenyl-2-ylmethyl)-2-methyl-1H-indol-4-yloxy)acetic acid, shown in FIG. 2 was synthesized. This compound is designated in these examples as ILY-4001, and is alternatively referred to herein as methyl indoxam.

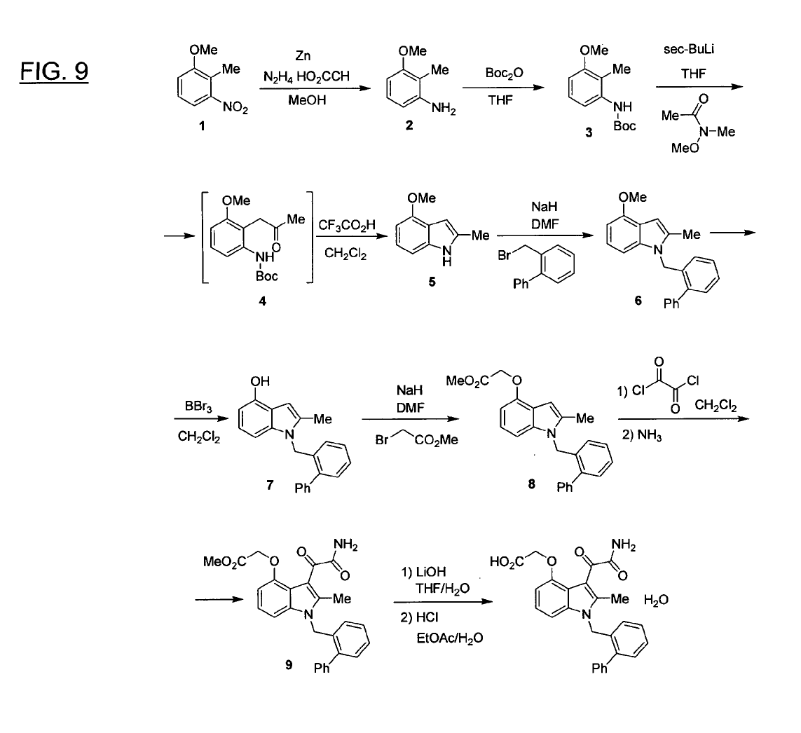

Reference is made to FIG. 9, which outlines the overall synthesis scheme for ILY-4001. The numbers under each compound shown in FIG. 9 correspond to the numbers in parenthesis associated with the chemical name for each compound in the following experimental description.

2-Methyl-3-methoxyaniline (2) [04-035-11]. To a stirred cooled (ca. 5° C.) hydrazine hydrate (159.7 g, 3.19 mol), 85% formic acid (172.8 g, 3.19 mol) was added drop wise at 10-20° C. The resultant mixture was added drop wise to a stirred suspension of zinc dust (104.3 g, 1.595 mol) in a solution of 2-methyl-3-nitroanisole (1) (53.34 g, 0.319 mol) in methanol (1000 mL). An exothermic reaction occurred. After the addition was complete, the reaction mixture was stirred for additional 2 h (until temperature dropped from 61° C. to RT) and the precipitate was filtered off and washed with methanol (3×150 mL). The filtrate was concentrated under reduced pressure to a volume of ca. 250 mL. The residue was treated with EtOAc (500 ml) and saturated aqueous NaHCO₃ (500 mL). The aqueous phase was separated off and discarded. The organic phase was washed with water (300 mL) and extracted with 1N HCl (800 mL). The acidic extract was washed with EtOAc (300 mL) and was basisified with K₂CO₃ (90 g). The free base 2 was extracted with EtOAc (3×200 mL) and the combined extracts were dried over MgSO₄. After filtration and removal of the solvent from the filtrate, product 2 was obtained as a red oil, which was used in the next step without further purification. Yield: 42.0 g (96%).

N-tert-Butyloxycarbonyl-2-methyl-3-methoxyaniline (3) [04-035-12]. A stirred solution of amine 2 (42.58 g, 0.31 mol) and di-tert-butyl dicarbonate (65.48 g, 0.30 mol) in THF (300 mL) was heated to maintain reflux for 4 h. After cooling to RT, the reaction mixture was concentrated under reduced pressure and the residue was dissolved in EtOAc (500 mL). The resultant solution was washed with 0.5 M citric acid (2×100 mL), water (100 mL), saturated aqueous NaHCO₃ (200 mL), brine (200 mL) and dried over MgSO₄. After filtration and removal of the solvent from the filtrate, the residue (red oil, 73.6 g) was dissolved in hexanes (500 mL) and filtered through a pad of Silica Gel (for TLC). The filtrate was evaporated under reduced pressure to provide N-Boc aniline 3 as a yellow solid. Yield: 68.1 g (96%).

4-Methoxy-2-methyl-1H-indole (5) [04-035-13]. To a stirred cooled (−50° C.) solution of N-Boc aniline 3 (58.14 g, 0.245 mol) in anhydrous THF (400 mL), a 1.4 M solution of sec-BuLi in cyclohexane (0.491 mol, 350.7 mL) was added drop wise at −48-−50° C. and the reaction mixture was allowed to warm up to −20° C. After cooling to −60° C., a solution of N-methoxy-N-methylacetamide (25.30 g, 0.245 mol) in THF (25 mL) was added drop wise at −57-−60° C. The reaction mixture was stirred for 1 h at −60° C. and was allowed to warm up to 15° C. during 1 h. After cooling to −15° C., the reaction was quenched with 2N HCl (245 mL) and the resultant mixture was adjusted to pH of ca. 7 with 2N HCl. The organic phase was separated off and saved. The aqueous phase was extracted with EtOAc (3×100 mL). The organic solution was concentrated under reduced pressure and the residual pale oil was dissolved in EtOAc (300 mL) and combined with the EtOAc extracts. The resultant solution was washed with water (2×200 mL), 0.5 M citric acid, (100 mL), saturated aqueous NaHCO₃ (100 mL), brine (200 mL) and dried over MgSO₄. After filtration and removal of the solvent from the filtrate, a mixture of starting N-Boc aniline 3 and intermediate ketone 4 (ca. 1:1 mol/mol) was obtained as a pale oil (67.05 g).

The obtained oil was dissolved in anhydrous CH₂Cl₂ (150 mL) and the solution was cooled to 0-−5° C. Trifluoroacetic acid (65 mL) was added drop wise and the reaction mixture was allowed to warm up to RT. After 16 h of stirring, an additional portion of trifluoroacetic acid (35 mL) was added and stirring was continued for 16 h. The reaction mixture was concentrated under reduced pressure and the red oily residue was dissolved in CH₂Cl₂ (500 mL). The resultant solution was washed with water (3×200 mL) and dried over MgSO₄. Filtration through a pad of Silica Gel 60 and evaporation of the filtrate under reduced pressure provided crude product 5 as a yellow solid (27.2 g). Purification by dry chromatography (Silica Gel for TLC, 20% EtOAc in hexanes) afforded indole 5 as a white solid. Yield: 21.1 g (53%)

1-[(1,1′-Biphenyl)-2-ylmethyl]-4-methoxy-2-methyl-1H-indole (6) [04-035-14]. A solution of indole 5 (16.12 g, 0.10 mol) in anhydrous DMF (100 mL) was added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture was stirred for 0.5 h at RT. After cooling the reaction mixture to ca. 5° C., 2-phenylbenzyl bromide (25.0 g, 0.101 mol) was added drop wise and the reaction mixture was stirred for 18 h at RT. The reaction was quenched with water (10 mL) and EtOAc (500 mL) was added. The resultant mixture was washed with water (2×200 mL+3×100 mL), brine (200 mL) and dried over MgSO₄. After filtration and removal of the solvent from the filtrate under reduced pressure, the residue (35.5 g, thick red oil) was purified by dry chromatography (Silica Gel for TLC, 5%→25% CH₂Cl₂ in hexanes) to afford product 6 as a pale oil. Yield: 23.71 g (72%).

1-[(1,1′-Biphenyl)-2-ylmethyl]-4-hydroxy-2-methyl-1H-indole (7) [04-035-15]. To a stirred cooled (ca. 10° C.) solution of the methoxy derivative 6 (23.61 g, 72.1 mmol) in anhydrous CH₂Cl₂ (250 mL), a 1M solution of BBr₃ in CH₂Cl₂ (300 mmol, 300 mL) was added drop wise at 15-20° C. and the dark reaction mixture was stirred for 5 h at RT. After concentrating of the reaction mixture under reduced pressure, the dark oily residue was cooled to ca. 5° C. and was dissolved in precooled (15° C.) EtOAc (450 mL). The resultant cool solution was washed with water (3×200 mL), brine (200 mL) and dried over MgSO₄. After filtration and removal of the solvent from the filtrate under reduced pressure, the residue (26.1 g, dark semi-solid) was purified by dry chromatography (Silica Gel for TLC, 5%→25% EtOAc in hexanes) to afford product 7 as a brown solid. Yield: 4.30 g (19%)

2-{1-[(1,1′-Biphenyl)-2-ylmethyl)-2-methyl-1H-indol-4-yl]oxy}-acetic acid methyl ester (8) [04-035-16]. To a stirred suspension of sodium hydride (0.549 g, 13.7 mmol, 60% in mineral oil) in anhydrous DMF (15 mL), a solution of compound 7 (4.30 g, 13.7 mmol) in DMF (30 mL) was added drop wise and the resultant mixture was stirred for 40 min at RT. Methyl bromoacetate (2.10 g, 13.7 mmol) was added drop wise and stirring was continued for 21 h at RT. The reaction mixture was diluted with EtOAc (200 mL) and washed with water (4×200 mL), brine (200 mL) and dried over MgSO₄. After filtration and removal of the solvent from the filtrate under reduced pressure, the residue (5.37 g, dark semi-solid) was purified by dry chromatography (Silica Gel for TLC, 5%→30% EtOAc in hexanes) to afford product 8 as a yellow solid. Yield: 4.71 g (89%).

2-{[3-(2-Amino-1,2-dioxoethyl)-1-[(1,1′-biphenyl)-2-ylmethyl)-2-methyl-1H-indol-4-yl]oxy}-acetic acid methyl ester (9) [04-035-17]. To a stirred solution of oxalyl chloride (1.55 g, 12.2 mmol) in anhydrous CH₂Cl₂ (20 mL), a solution of compound 8 in CH₂Cl₂ (40 mL) was added drop wise and the reaction mixture was stirred for 80 min at RT. After cooling the reaction mixture to −10° C., a saturated solution of NH₃ in CH₂Cl₂ (10 mL) was added drop wise and then the reaction mixture was saturated with NH₃ (gas) at ca. 0° C. Formation of a precipitate was observed. The reaction mixture was allowed to warm up to RT and was concentrated under reduced pressure to dryness. The dark solid residue (6.50 g) was subjected to dry chromatography (Silica Gel for TLC, 30% EtOAc in hexanes→100% EtOAc) to afford product 9 as a yellow solid. Yield: 4.64 g (83%).

2-{[3-(2-Amino-12-dioxoethyl)-1-[(1,1′-biphenyl)-2-ylmethyl)-2-methyl-1H-indol-4-yl]oxy}-acetic acid (ILY-4001) [04-035-18]. To a stirred solution of compound 9 (4.61 g, 10.1 μmol) in a mixture of THF (50 mL) and water (10 mL), a solution of lithium hydroxide monohydrate (0.848 g, 20.2 mmol) in water (20 mL) was added portion wise and the reaction mixture was stirred for 2 h at RT. After addition of water (70 mL), the reaction mixture was concentrated under reduced pressure to a volume of ca. 100 mL. Formation of a yellow precipitate was observed. To the residual yellow slurry, 2N HCl (20 mL) and EtOAc (200 mL) were added and the resultant mixture was stirred for 16 h at RT. The yellowish-greenish precipitate was filtered off and washed with EtOAc (3×20 mL), Et₂O (20 mL) and hexanes (20 mL). After drying in vacuum, the product (2.75 g) was obtained as a pale solid. MS: 443.27 (M⁺+1). Elemental Analysis Calcd for C₂₆H₂₂N₂O₅+H₂O: C, 67.82; H, 5.25; N, 6.08. Found: C, 68.50; H, 4.96; N, 6.01. HPLC: 96.5% purity. ¹H NMR (DMSO-d₆) δ7.80 (br s, 1H), 7.72-7.25 (m, 9H), 7.07 (t, 1H), 6.93 (d, 1H), 6.57 (d, 1H), 6.43 (d, 1H), 5.39 (s, 2H), 4.68 (s, 2H), 2.38 (s, 3H).

The aqueous phase of the filtrate was separated off and the organic one was washed with brine (100 mL) and dried over MgSO₄. After filtration and removal of the solvent from the filtrate under reduced pressure, the greenish solid residue was washed with EtOAc (3×10 mL), Et₂O (10 mL) and hexanes (10 mL). After drying in vacuum, an additional portion (1.13 g) of product was obtained as a greenish solid. Total yield: 2.75 g+1.13 g=3.88 g (87%).

### Example 5

### In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor and for Treatment of Diet-Related Conditions

This example demonstrated that the compound 2-(3-(2-amino-2-oxoacetyl)-1-(biphenyl-2-ylmethyl)-2-methyl-1H-indol-4-yloxy)acetic acid, shown in FIG. 2, was an effective phospholipase-2A IB inhibitor, with phenotypic effects approaching and/or comparable to the effect of genetically deficient PLA2 (−/−) mice. This example also demonstrated that this compound is effective in treating conditions such as weight-related conditions, insulin-related conditions, and cholesterol-related conditions, including in particular conditions such as obesity, diabetes mellitus, insulin resistance, glucose intolerance, hypercholesterolemia and hypertriglyceridemia. In this example, the compound 2-(3-(2-amino-2-oxoacetyl)-1-(biphenyl-2-ylmethyl)-2-methyl-1H-indol-4-yloxy)acetic acid is designated as ILY-4001 (and is alternatively referred to herein as methyl indoxam).

ILY-4001 (FIG. 2) was evaluated as a PLA2 IB inhibitor in a set of experiments using wild-type mice and genetically deficient PLA2 (−/−) mice (also referred to herein as PLA2 knock-out (KO) mice). In these experiments, wild-type and PLA2 (−/−) mice were maintained on a high fat/high sucrose diet, details of which are described below.

ILY-4001 has a measured IC50 value of around 0.2 uM versus the human PLA2 IB enzyme and 0.15 uM versus the mouse PLA2 IB enzyme, in the context of the 1-palmitoyl-2-(10-pyrenedecanoyl)-sn-glycero-3-phosphoglycerol assay, which measures pyrene substrate release from vesicles treated with PLA2 IB enzyme (Singer, Ghomashchi et al. 2002). An IC-50 value of around 0.062 was determined in experimental studies. (See Example 6A). In addition to its activity against mouse and human pancreatic PLA2, methyl indoxam is stable at low pH, and as such, would be predicted to survive passage through the stomach. ILY-4001 has relatively low absorbtion from the GI lumen, based on Caco-2 assays (See Example 6B), and based on pharmokinetic studies (See Example 6C).

In the study of this Example 5, twenty-four mice were studied using treatment groups as shown in Table 4, below. Briefly, four groups were set up, each having six mice. Three of the groups included six wild-type PLA2 (+/+) mice in each group (eighteen mice total), and one of the groups included six genetically deficient PLA2 (−/−) mice. One of the wild-type groups was used as a wild-type control group, and was not treated with ILY-4001. The other two wild-type groups were treated with ILY-4001—one group at a lower dose (indicated as “L” in Table 1) of 25 mg/kg/day, and the other at a higher dose (indicated as “H” in Table 1) of 90 mg/kg/day. The group comprising the PLA2 (−/−) mice was used as a positive control group.

TABLE 4
Treatment Groups for ILY-4001 Study

| | | | ILY-4001 | |
|---|---|---|---|---|
| Group | Treatment | Number | Dose Levels | Duration |
| Number | Groups | of Animals | (mg/kg/day) | (weeks) |
| 1 | C57BL/6(wt) | 6 | 0 | 10 |
| 2 | C57BL/6(wt) | 6 | 25 (L) | 10 |
| 3 | C57BL/6(wt) | 6 | 90 (H) | 10 |
| 4 | C57BL/6(PLA₂- | 6 | 0 | 10 |
| | KO) | | | |

The experimental protocol used in this study was as follows. The four groups of mice, including wild type and isogenic PLA2 (−/−) C57BL/J mice were acclimated for three days on a low fat/low carbohydrate diet. After the three day acclimation phase, the animals were fasted overnight and serum samples taken to establish baseline plasma cholesterol, triglyceride, and glucose levels, along with baseline body weight. The mice in each of the treatment groups were then fed a high fat/high sucrose diabetogenic diet (Research Diets D12331). 1000 g of the high fat/high sucrose D12331 diet was composed of casein (228 g), DL-methionine (2 g), maltodextrin 10 (170 g), sucrose (175 g), soybean oil (25 g), hydrogenated coconut oil (333.5 g), mineral mix S10001 (40 g), sodium bicarbonate (10.5 g), potassium citrate (4 g), vitamin mix V10001 (10 g), and choline bitartrate (2 g). This diet was supplemented with ILY-4001 treatments such that the average daily dose of the compound ingested by a 25 g mouse was: 0 mg/kg/day (wild-type control group and PLA2 (−/−) control group); 25 mg/kg/day (low-dose wild-type treatment group), or 90 mg/kg/day (high-dose wild-type treatment group). The animals were maintained on the high fat/high sucrose diet, with the designated ILY-4001 supplementation, for a period of ten weeks.

Body weight measurements were taken for all animals in all treatment and control groups at the beginning of the treatment period and at 4 weeks and 10 weeks after initiation of the study. (See Example 5A). Blood draws were also taken at the beginning of the treatment period (baseline) and at 4 weeks and 10 weeks after initiation of the study, in order to determine fasting glucose (See Example 5B). Cholesterol and triglyceride levels were determined from blood draws taken at the beginning of the treatement (baseline) and at ten weeks. (See Example 5C).

### Example 5A

### Body-Weight Gain in In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor

In the study generally described above in Example 5, body weight measurements were taken for all animals in all treatment and control groups at the beginning of the treatment period and at 4 weeks and 10 weeks after initiation of the study. Using the treatment protocol described above with ILY-4001 supplemented into a high fat/high sucrose diabetogenic diet, notable decreases were seen in body weight gain.

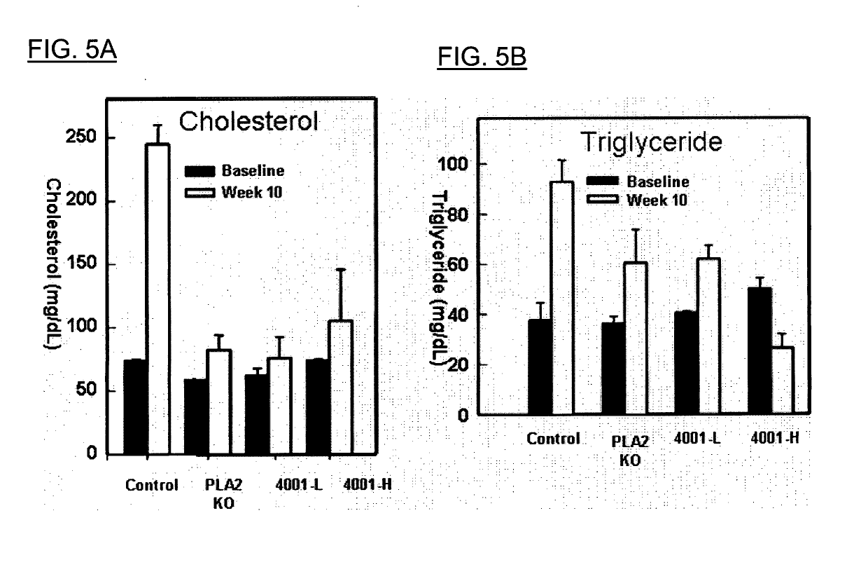

With reference to FIG. 3, body weight gain in the wild-type mice receiving no ILY-4001 (group 1, wild-type control) followed the anticipated pattern of a substantial weight gain from the beginning of the study to week 4, and a further doubling of weight gain by week 10. In contrast, body weight gain for the PLA2 (−/−) mice (PLA2 KO mice) also receiving no ILY-4001 and placed on the same diet (group 4, PLA2 (−/−) control) did not show statistically significant changes from week 4 to week 10, and only a marginal increase in body weight over the extent of the study (<5 g). The two treatment groups (25 mg/kg/d and 90 mg/kg/d) showed significantly reduced body weight gains at week 4 and week 10 of the study compared to the wild-type control group. Both treatment groups showed body weight gain at four weeks modulated to an extent approaching that achieved in the PLA2 (−/−) mice. The low-dose treatment group showed body weight gain at ten weeks modulated to an extent comparable to that achieved in the PLA2 (−/−) mice.

### Example 5B

### Fasting Serum Glucose in In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor

In the study generally described above in Example 5, blood draws were taken at the beginning of the treatment period (baseline) and at 4 weeks and 10 weeks after initiation of the study, in order to determine fasting glucose. Using the treatment protocol described above with ILY-4001 supplemented into a high fat/high sucrose diabetogenic diet, notable decreases were seen in fasting serum glucose levels.

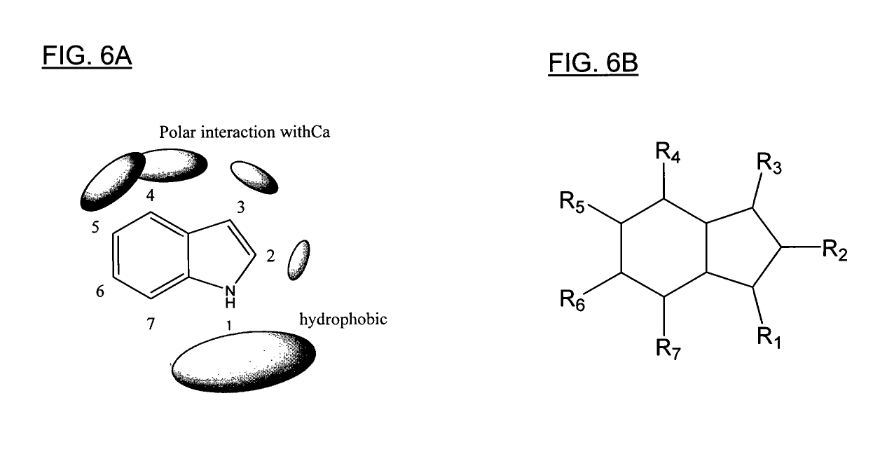

Referring to FIG. 4, the wild-type control mice (group 1) showed a sustained elevated plasma glucose level, consistent with and indicative of the high fat/high sucrose diabetogenic diet at both four weeks and ten weeks. In contrast, the PLA2 (−/−) KO mice (group 4) showed a statistically significant decrease in fasting glucose levels at both week 4 and week 10, reflecting an increased sensitivity to insulin not normally seen in mice placed on this diabetogenic diet. The high dose ILY-4001 treatment group (group 3) showed a similar reduction in fasting glucose levels at both four weeks and ten weeks, indicating an improvement in insulin sensitivity for this group as compared to wild-type mice on the high fat/high sucrose diet, and approaching the phenotype seen in the PLA2 (−/−) KO mice. In the low dose ILY-4001 treatment group (group 2), a moderately beneficial effect was seen at week four; however, a beneficial effect was not observed at week ten.

### Example 5C

### Serum Cholesterol and Triglycerides in In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor

In the study generally described above in Example 5, blood draws were taken at the beginning of the treatment period (baseline) and at 10 weeks after initiation of the study, in order to determine cholesterol and triglyceride levels. Using the treatment protocol described above with ILY-4001 supplemented into a high fat/high sucrose diabetogenic diet, notable decreases were seen in both serum cholesterol levels and serum triglyceride levels.

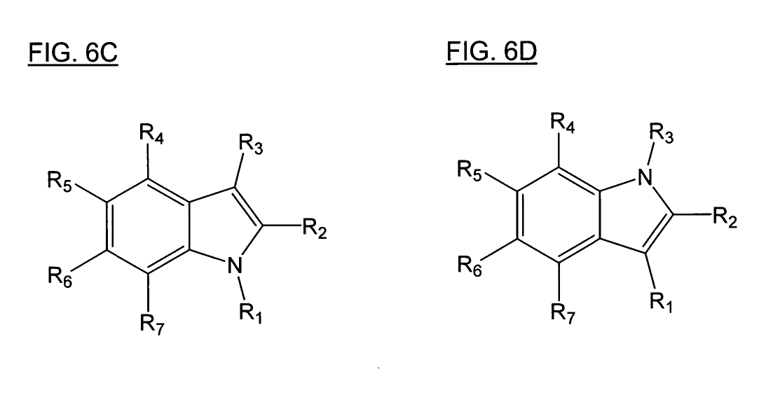

With reference to FIGS. 5A and 5B, after 10 weeks on the high fat/high sucrose diet, the wild-type control animals (group 1) had notable and substantial increases in both circulating cholesterol levels (FIG. 5A) and triglyceride levels (FIG. 5B), relative to the baseline measure taken at the beginning of the study. The PLA2 (−/−) KO animals (group 4), in contrast, did not show the same increase in these lipids, with cholesterol and triglyceride values each 2 to 3 times lower than those found in the wild-type control group. Significantly, treatment with ILY-4001 at both the low and high doses (groups 2 and 3, respectively) substantially reduced the plasma levels of cholesterol and triglycerides, mimicking the beneficial effects at levels comparable to the PLA2 (−/−) KO mice.

### Example 6

### Characterization Studies—ILY-4001 [2-(3-(2-amino-2-oxoacetyl)-1-(biphenyl-2-ylmethyl)-2-methyl-1H-indol-4-yloxy)acetic acid]

This example characterized ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, with respect to activity, as determined by IC50 assay (Example 6A), with respect to cell absorbtion, as determined by in-vitro Caco-2 assay (Example 6B) and with respect to bioavailability, as determined using in-vivo mice studies (Example 6C).

### Example 6A

### IC-50 Study—ILY-4001 [2-(3-(2-amino-2-oxoacetyl)-1-(biphenyl-2-ylmethyl)-2-methyl-1H-indol-4-yloxy)acetic acid]

This example evaluated the IC50 activity value of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam.

A continuous fluorimetric assay for PLA2 activity described in the literature was used to determine IC (Leslie, C C and Gelb, M H (2004) Methods in Molecular Biology “Assaying phospholipase A2 activity”, 284: 229-242, Singer, A G, et al. (2002) Journal of Biological Chemistry “Interfacial kinetic and binding properties of the complete set of human and mouse groups I, II, V, X, and XII secreted phospholipases A2”, 277: 48535-48549, Bezzine, S, et al. (2000) Journal of Biological Chemistry “Exogenously added human group X secreted phospholipase A(2) but not the group IB, IIA, and V enzymes efficiently release arachidonic acid from adherent mammalian cells”, 275: 3179-3191) and references therein.

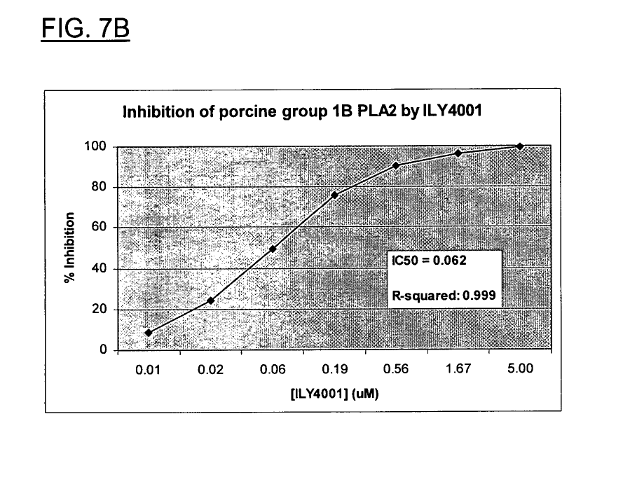

Generally, this assay used a phosphatidylglycerol (or phosphatidylmethanol) substrate with a pyrene fluorophore on the terminal end of the sn-2 fatty acyl chain. Without being bound by theory, close proximity of the pyrenes from neighboring phospholipids in a phospholipid vesicle caused the spectral properties to change relative to that of monomeric pyrene. Bovine serum albumin was present in the aqueous phase and captured the pyrene fatty acid when it is liberated from the glycerol backbone owing to the PLA2-catalyzed reaction. In this assay, however, a potent inhibitor can inhibit the liberation of pyrene fatty acid from the glycerol backbone. Hence, such features allow for a sensitive PLA2 inhibition assay by monitoring the fluorescence of albumin-bound pyrene fatty acid, as represented in Scheme 1 shown in FIG. 7A. The effect of a given inhibitor and inhibitor concentration on any given phospholipase can be determined.

In this example, the following reagents and equipment were obtained from commercial vendors:
1. Porcine PLA2 IB
2. 1-hexadecanoyl-2-(1-pyrenedecanoyl)-sn-glycero-3-phosphoglycerol (PPyrPG)
3. 1-hexadecanoyl-2-(1-pyrenedecanoyl)-sn-glycero-3-phosphomethanol (PPyrPM)
4. Bovine serum albumin (BSA, fatty acid free)
5. 2-Amino-2-(hydroxymethyl)-1,3-propanediol, hydrochloride (Tris-HCl)
6. Calcium chloride
7. Potassium chloride
8. Solvents: DMSO, toluene, isopropanol, ethanol
9. Molecular Devices SPECTRAmax microplate spectrofluorometer
10. Costar 96 well black wall/clear bottom plate

In this example, the following reagents were prepared:

1. PPyrPG (or PPyrPM) stock solution (1 mg/ml) in toluene:isopropanol (1:1)

2. Inhibitor stock solution (10 mM) in DMSO

3. 3% (w/v) bovine serum albumin (BSA)

4. Stock buffer: 50 mM Tris-HCl, pH 8.0, 50 mM KCl and 1 mM CaCl₂

In this example, the procedure was performed as follows:
1. An assay buffer was prepared by adding 3 ml 3% BSA to 47 ml stock buffer.
2. Solution A was prepared by adding serially diluted inhibitors to the assay buffer. Inhibitor were three-fold diluted in a series of 8 from 15 uM.
3. Solution B was prepared by adding PLA2 to the assay buffer. This solution was prepared immediately before use to minimize enzyme activity loss.
4. Solution C was prepared by adding 30 ul PPyrPG stock solution to 90 ul ethanol, and then all 120 ul of PPyrPG solution was transferred drop-wise over approximately 1 min to the continuously stirring 8.82 ml assay buffer to form a final concentration of 4.2 uM PPyrPG vesicle solution.
5. The SPECTRAmax microplate spectrofluorometer was set at 37° C.
6. 100 ul of solution A was added to each inhibition assay well of a costar 96 well black wall/clear bottom plate
7. 100 ul of solution B was added to each inhibition assay well of a costar 96 well black wall/clear bottom plate.
8. 100 ul of solution C was added to each inhibition assay well of a costar 96 well black wall/clear bottom plate.
9. The plate was incubated inside the spectrofluorometer chamber for 3 min.
10. The fluorescence was read using an excitation of 342 nm and an emission of 395 nm.

In this example, the IC50 was calculated using the BioDataFit 1.02 (Four Parameter Model) software package. The equation used to generate the curve fit is:

$${y}_{j} = β + \frac{α - β}{1 + exp ( - κ ( log ( {x}_{j} ) - γ ) )}$$

wherein: α is the value of the upper asymptote; β is the value of the lower asymptote; κ is a scaling factor; γ is a factor that locates the x-ordinate of the point of inflection at

$$exp [ \frac{κγ - log ( \frac{1 + κ}{κ - 1} )}{κ} ]$$

with constraints α, β, κ, γ>0, β<α, and β<γ<α.

The results, shown in FIG. 7B, indicate that the concentration of ILY4001 resulting in 50% maximal PLA2 activity was calculated to be 0.062 uM.

### Example 6B

### Caco-2 Absorbtion Study—ILY-4001 [2-(3-(2-amino-2-oxoacetyl)-1-(biphenyl-2-ylmethyl)-2-methyl-1H-indol-4-yloxy)acetic acid]

This example evaluated the intestinal absorption of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam using in-vitro assays with Caco-2 cells.

Briefly, the human colon adenocarcinoma cell line, Caco-2, was used to model intestinal drug absorption. It has been shown that the apparent permeability values measured in Caco-2 monolayers in the range of 1×10⁻⁷ cm/sec or less typically correlate with relatively poor human absorption. (Artursson, P., K. Palm, et al. (2001). “Caco-2 monolayers in experimental and theoretical predictions of drug transport.” Adv Drug Deliv Rev 46(1-3): 27-43.)

In order to determine the compound permeability, Caco-2 cells (ATCC) were seeded into 24-well transwells (Costar) at a density of 6×10⁴ cells/cm². Monolayers were grown and differentiated in MEM (Mediatech) supplemented with 20% FBS, 100 U/ml penicillin, and 100 ug/ml streptomycin at 37° C., 95% humidity, 95% air, and 5% CO₂. The culture medium was refreshed every 48 hours. After 21 days, the cells were washed in transport buffer made up of HBSS with HEPES and the monolayer integrity was evaluated by measuring the trans-epithelial electrical resistance (TEER) of each well. Wells with TEER values of 350 ohm-cm² or better were assayed.

ILY-4001 and Propranolol (a transcellular transport control) were diluted to 50 ug/ml in transport buffer and added to the apical wells separately. 150 ul samples were collected for LC/MS analysis from the basolateral well at 15 min, 30 min, 45 min, 1 hr, 3 hr, and 6 hr time points; replacing the volume with pre-warmed transport buffer after each sampling. The apparent permeabilities in cm/s were calculated based on the equation:
Pₐₚₚ=(dQ/dt)×(1/C₀)×(1/A)
Where dQ/dt is the permeability rate corrected for the sampling volumes over time, C₀ is the initial concentration, and A is the surface area of the monolayer (0.32 cm²). At the end of the experiment, TEER measurements were retaken and wells with readings below 350 ohm-cm² indicated diminished monolayer integrity such that the data from these wells were not valid for analysis. Finally, wells were washed with transport buffer and 100 uM of Lucifer Yellow was added to the apical wells. 15 min, 30 min, and 45 min time points were sampled and analyzed by LC/MS to determine paracellular transport.

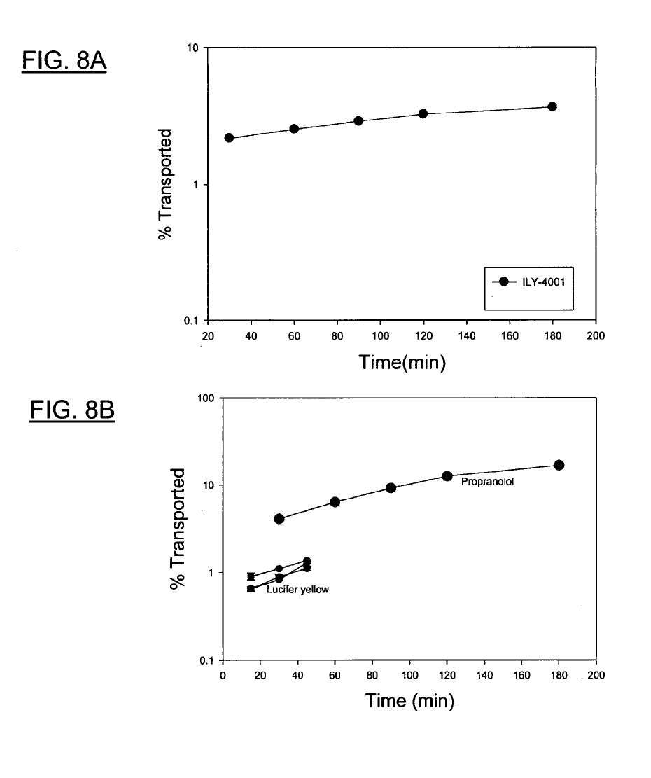

Results from the Caco-2 permeability study for ILY-4001 are shown in FIG. 8A, in which the apparent permeability (cm/s) for ILY-4001 was determined to be around 1.66×10⁻⁷. The results for Lucifer Yellow and Propranolol permeability as paracellular and transcellular transport controls were also determined, and are shown in FIG. 8B, with determined apparent permeability (cm/s) of around 1.32×10⁻⁵ for Propranolol and around 2.82×10⁻⁷+/−0.37×10⁻⁷ for Lucifer Yellow.

### Example 6C

### Pharmokinetic Study—ILY-4001 [2-(3-(2-amino-2-oxoacetyl)-1-(biphenyl-2-ylmethyl)-2-methyl-1H-indol-4-yloxy)acetic acid] (Methyl Indoxam)

This example evaluated the bioavailability of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam. Specifically, a pharmokinetic study was conducted to determine the fraction of unchanged ILY-4001 in systemic circulation following administration.

Bioavailability was calculated as a ratio of AUC-oral/AUC-intravenous (IV). To determine this ratio, a first set of subject animals were given a measured intravenous (IV) dose of ILY-4001, followed by a determination of ILY-4001 levels in the blood at various time points after administration (e.g., 5 minutes through 24 hours). Another second set of animals was similarly dosed using oral administration, with blood levels of ILY-4001 determined at various time points after administration (e.g., 30 minutes through 24 hours). The level of ILY-4001 in systemic circulation were determined by generally accepted methods (for example as described in Evans, G., A Handbook of Bioanalysis and Drug Metabolism. Boca Raton, CRC Press (2004)). Specifically, liquid scintillation/mass spectrometry/mass spectrometry (LC/MS/MS) analytical methods were used to quantitate plasma concentrations of ILY-4001 after oral and intravenous administration. Pharmacokinetic parameters that were measured include Cₘₐₓ, AUC, tₘₐₓ, t_{1/2}, and F (bioavailability).

In this procedure, ILY-4001 was dosed at 3 mg/kg IV and 30 mg/kg oral. The results of this study, summarized in Table 5, showed a measured bioavailability of 28% of the original oral dose. This indicated about a 72% level of non-absorption of ILY-4001 from the GI tract into systemic circulation.

TABLE 5
Results of Pharmokinetic Study for ILY-4001

| | IV | ORAL |
|---|---|---|
| t½ (h) | 1.03 | 1.25 |
| Cmax (ng/mL) | 3168 | 2287 |
| Tmax (h) | 0.083 | 1 |
| AUC 0-24) (h * ng/mL) | 2793 | 5947 |
| AUC(0-inf) (h * ng/mL) | 2757 | 5726 |
| % F | | 28.0 |

All publications, patents, and patent applications mentioned in this specification are herein incorporated by reference to the same extent as if each individual publication, patent, or patent application was specifically and individually indicated to be incorporated by reference.

It can be appreciated to one of ordinary skill in the art that many changes and modifications can be made thereto without departing from the spirit or scope of the appended claims, and such changes and modifications are contemplated within the scope of the instant invention.

1.-59. (canceled)

60. A method for modulating serum non-HDL cholesterol in a subject, the method comprising the steps of identifying the subject as a member of a population at risk of one or more of obesity, insulin resistance or diabetes mellitus and administering an effective amount of a phospholipase-A₂ IB inhibitor to the subject.

61. A method for modulating serum triglyceride in a subject, the method comprising the steps of identifying the subject as a member of a population at risk of one or more of obesity, insulin resistance or diabetes mellitus and administering an effective amount of a phospholipase-A₂ IB inhibitor to the subject.

62. A method of treating a condition in a subject, the condition being selected from the group consisting of hypercholesterolemia, hypertrigliceridemia, atherosclerosis, coronary artery disease and combinations thereof, the method comprising the steps of identifying the subject as a member of a population at risk of one or more of obesity, insulin resistance or diabetes mellitus and administering an effective amount of a phospholipase-A₂ IB inhibitor to the subject.

63. The method of claim 62 wherein the condition is hypercholesterolemia.

64. The method of claim 62 wherein the condition is hypertriglyceridemia.

65. The method of claim 62 wherein the condition is atherosclerosis.

66. The method of claim 62 wherein the condition is coronary artery disease.

67. The method of claim 62 wherein the treatment is prophylactic.

68. The method of claim 62 wherein the treatment is therapeutic.

69. The method of claim 62 wherein the subject is a member of a population at risk for diabetes type 2.

70. The method of any of claims 60 through 62 wherein the phospholipase-A₂ IB inhibitor comprises a substituted organic compound having a fused five-member ring and six-member ring, or a salt thereof.

71. The method of any of claims 60 through 62 wherein the phospholipase-A₂ IB inhibitor comprises a fused five-member ring and six-member ring having one or more heteroatoms substituted within the ring structure of the five-member ring, within the ring structure of the six-member ring, or within the ring structure of each of the five-member and six-member rings, or a salt thereof.

72. The method of claim 70 wherein the phospholipase-A₂ IB inhibitor comprises a compound, or a salt thereof represented by the formula 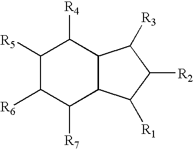
wherein the fused five-membered-ring and six-membered-ring core structure can be saturated or unsaturated, and wherein R₁ through R₇ are independently selected from the group consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

73. The method of claim 72 wherein R₁ through R₇ can independently comprise, independently selected additional rings between two adjacent substitutents, such additional rings being independently selected 5-, 6-, and/or 7-member rings which are carbocyclic rings, heterocyclic rings, and combinations thereof.

74. The method of any of claims 60 through 62 wherein the phospholipase-A₂ IB inhibitor comprises an indole compound, or a salt thereof.

75. The method of any of claims 60 through 62 wherein the phospholipase-A₂ IB inhibitor comprises an indole compound, or a salt thereof, selected from the formulas 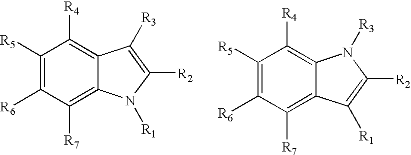
wherein with respect to each of the formulas, R₁ through R₇ are independently selected from the groups consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

76. The method of claim 75 wherein with respect to each of the formulas, R₁ through R₇ can independently comprise, independently selected additional rings between two adjacent substitutents, such additional rings being independently selected 5-, 6-, and/or 7-member rings which are carbocyclic rings, heterocyclic rings, and combinations thereof.

77. The method of claim 75 wherein
R₁ is selected from the group consisting of hydrogen, oxygen, sulfur, amine, halide, hydroxyl (—OH), thiol (—SH), carbonyl, acidic, alkyl, alkenyl, carbocyclic, heterocyclic, and substituted substitution group;
R₂ is selected from the group consisting of hydrogen, oxygen, halide, carbonyl, alkyl, alkenyl, carbocyclic, and substituted substitution group;
R₃ is selected from the group consisting of hydrogen, oxygen, sulfur, amine, hydroxyl (—OH), thiol (—SH), carbonyl, acidic, alkyl, heterocyclic, acylamino, oximyl, hydrazyl, and substituted substitution group;
R₄ and R₅ are each independently selected from the group consisting of hydrogen, oxygen, sulfur, phosphorus, amine, hydroxyl (—OH), thiol (—SH), carbonyl, acidic, alkyl, alkenyl, heterocyclic, acylamino, oximyl, hydrazyl, and substituted substitution group;
R₆ is selected from the group consisting of hydrogen, oxygen, amine, halide, hydroxyl (—OH), acidic, alkyl, carbocyclic, acylamino and substituted substitution group; and
R₇ is selected from the groups consisting of hydrogen, halide, thiol (—SH), carbonyl, acidic, alkyl, alkenyl, carbocyclic, and substituted substitution group.

78. The method of any of claims 60 through 62 wherein the phospholipase-A₂ IB inhibitor is localized in a gastrointestinal lumen upon administration or ingestion.

79. The method of any of claims 60 through 62 wherein the phospholipase-A₂ IB inhibitor inhibits activity of secreted, calcium-dependent phospholipase-A₂ IB present in the gastrointestinal lumen.

80. The method of claim 60 wherein the phospholipase-A₂ IB inhibitor inhibits activity of pancreas-secreted phospholipase-A₂ IB.

81. The method of claim 60 wherein the effective amount of the phospholipase-A2 IB inhibitor is an amount sufficient to inhibit at least about 30% of phospholipase-A2 IB activity.

82. The method of claim 60 wherein the phospholipase-A₂ inhibitor essentially does not inhibit a lipase.

83. The method of claim 60 wherein the phospholipase-A₂ inhibitor essentially does not inhibit phospholipase-B.

84. The method of claim 60 wherein the phospholipase inhibitor inhibits activity of phospholipase A₂, but essentially does not inhibit other gastrointestinal phospholipases having activity for catabolizing a phospholipid.

85. The method of claim 60 wherein the phospholipase inhibitor inhibits activity of phospholipase A₂, but essentially does not inhibit other gastrointestinal phospholipases having activity for catabolizing phosphatidylcholine or phosphatidylethanolamine.

86. The method of claims 60 wherein the phospholipase inhibitor inhibits activity of phospholipase A₂, but essentially does not inhibit other gastrointestinal mucosal membrane-bound phospholipases.

87. The method of any of claims 60 through 62 wherein the subject is a member of a population at risk of obesity or insulin resistance induced by high fat and high carbohydrate diet.

88. The method of any of claims 60 through 62 wherein the subject is a member of a population at risk of obesity or insulin resistance induced by high fat and high saccharide diet.

89. The method of any of claims 60 through 62 wherein the subject is also a member of a population at risk of cardiovascular disease.

90. A food product composition comprising an edible foodstuff and a phospholipase-A₂ IB inhibitor.
